SEQUENCE LISTING (53 sequences)

SEQ ID NO: 1 - DNA, 375 bp, Artificial Sequence
  3B9 VL1-VH nucleic acid sequence
caggtgcagctggtgcagtctggggctgaggtgaagaagcctgggtcctcggtgaaggtctcctgcaaggcttctggaggcaccttcagcagctatgctatcagctgggtgcgccaggcccctggacaagggcttgagtggatgggagggatcatccctctctttgatacaacaaagtacgcacagcagttccagggcagagtcacgattaccgcggacgaatccacgagcacagcctacatggagctgagcagcctgagatctgaggacacggccgtattttactgtgcgagagatcgggatattttgactgattattatcccatgggcggtatggacgtctggggccaagggaccacggtcaccgtctcctca

SEQ ID NO: 2 - protein, 125 aa, Artificial Sequence
  3B9 VL1-VH amino acid sequence
QVQLVQSGAEVKKPGSSVKVSCKASGGTFSSYAISWVRQAPGQGLEWMGGIIPLFDTTKYAQQFQGRVTITADESTSTAYMELSSLRSEDTAVFYCARDRDILTDYYPMGGMDVWGQGTTVTVSS

SEQ ID NO: 3 - DNA, 324 bp, Artificial Sequence
  39B9 VL1-VL nucleic acid sequence
gccatccagttgacccagtctccatcctccctgtctgcatctgtaggagacagagtcaccatcacttgccgggcaagtcagggcattagcagtgttttagcctggtatcagcagaaaccagggaaagctcctaagctcctgatctatgatgcctccagtttggaaagtggggtcccatcaaggttcagcggcagtggatctgggacagatttcactctcaccatcagcagcctgcagcctgaagattttgcaacttattactgtcaacagtctaatagttacccgctcactttcggcggagggaccaaggtggagatcaaacgt

SEQ ID NO: 4 - protein, 108 aa, Artificial Sequence
  39B9 VL1-VL amino acid sequence
AIQLTQSPSSLSASVGDRVTITCRASQGISSVLAWYQQKPGKAPKLLIYDASSLESGVPSRFSGSGSGTDFTLTISSLQPEDFATYYCQQSNSYPLTFGGGTKVEIKR

SEQ ID NO: 5 - DNA, 324 bp, Artificial Sequence
  39B9 VL5-VL nucleic acid sequence
gacatcctgatgacccagtctccatcctccctgtctgcatctgtaggagacagagtcaccatcacttgtcgggcgagtcaggatattagcagctggttagcctggtatcagcagaaaccagggaaagctcctaagctcctgatctatgatgcctccagtttggaaagtggggtcccatcaaggttcagcggcagtggatctgggacagatttcactctcaccatcagcagcctgcagcctgaagattttgcaacttattactgtcaacagtctaatagttacccgctcactttcggcggagggaccaaggtggagatcaaacga

SEQ ID NO: 6 - protein, 108 aa, Artificial Sequence
  39B9 VL5-VL amino acid sequence
DILMTQSPSSLSASVGDRVTITCRASQDISSWLAWYQQKPGKAPKLLIYDASSLESGVPSRFSGSGSGTDFTLTISSLQPEDFATYYCQQSNSYPLTFGGGTKVEIKR

SEQ ID NO: 7 - DNA, 357 bp, Artificial Sequence
  12A VH nucleic acid sequence
caggtgcagctggtggagtcttggggaggcgtggtccagcctgggaggtccctgagactctcctgtgcagcgtctggattcaccttcagtaactatgacatgtactgggtccgccaggctccaggcaaggggctggagtgggtggcagttatattagatgatggaaataataattactacgcagactccgtgaagggccgattcaccatctccagagacaattccaagaaaaaggtgtatctgcaaatgaatagcctgagagctgaggacacggctgtgtattactgtgtgagagcgtcccctaactggggtcttcttgacttctggggccagggaaccctggtcaccgtctcgagt

SEQ ID NO: 8 - protein, 119 aa, Artificial Sequence
  12A VH amino acid sequence
QVQLVESWGGVVQPGRSLRLSCAASGFTFSNYDMYWVRQAPGKGLEWVAVILDDGNNNYYADSVKGRFTISRDNSKKKVYLQMNSLRAEDTAVYYCVRASPNWGLLDFWGQGTLVTVSS

SEQ ID NO: 9 - DNA, 324 bp, Artificial Sequence
  12A VL nucleic acid sequence
gaaattgtgttgacacagtctccatcctcactgtctgcatctgtaggagacagagtcaccatcacttgtcgggcgagtcagggtattagcagctggttagcctggtatcagcagaaaccagggaaagctcctaagctcctgatctatgatgcctccagtttggaaagtggggtcccatcaaggttcagcggcagtggatctgggacagatttcactctcaccatcagcagcctgcagcctgaagattttgcaacttattactgtcaacagtttaatagttacccgatcaccttcggccaagggacacgactggagattaaacgt

SEQ ID NO: 10 - protein, 108 aa, Artificial Sequence
  12A VL amino acid sequence
EIVLTQSPSSLSASVGDRVTITCRASQGISSWLAWYQQKPGKAPKLLIYDASSLESGVPSRFSGSGSGTDFTLTISSLQPEDFATYYCQQFNSYPITFGQGTRLEIKR

SEQ ID NO: 11 - DNA, 357 bp, Artificial Sequence
  5C VH nucleic acid sequence
caggtgcagctggtgcagtctgggggaggcgtggtccagcctgggaggtccctgagactctcctgtgcagcctctggattcatcttcagtagctatgacatgtactgggtccgccaggctccaggcaaggggctggagtgggtggcagttatattatatgatggaaataataaatactacgcagactccgtgaagggccgattcaccatctccagagacaattccaagaacacggtgtatctgcaaatgaacagcctgagagctgaggacacggctgtgtattactgtgtgagagcgtcccctaactggggtctttttgacttctggggccagggaaccctggtcaccgtctcgagt

SEQ ID NO: 12 - protein, 119 aa, Artificial Sequence
  5C VH amino acid sequence
QVQLVQSGGGVVQPGRSLRLSCAASGFIFSSYDMYWVRQAPGKGLEWVAVILYDGNNKYYADSVKGRFTISRDNSKNTVYLQMNSLRAEDTAVYYCVRASPNWGLFDFWGQGTLVTVSS

SEQ ID NO: 13 - DNA, 324 bp, Artificial Sequence
  5C VL nucleic acid sequence
gacatccagatgacccagtctccatcctccctgtctgcatctgtaggagacagagtcaccatcacttgccgggcaagtcagggcattagcagtgatttagcctggtatcagcagaaaccagggaaagctcctaagctcctgatgtatgatgcctccagtttggaaagtggggtcccatcaaggttcagcggcagtggatctgggacagatttcactctcaccatcagcagcctgcagcctgaagattttgcaacttattactgtcaacagtttaatagttacccgatcaccttcggccaagggacacgactggagattaaacgt

SEQ ID NO: 14 - protein, 108 aa, Artificial Sequence
  5C VL amino acid sequence
DIQMTQSPSSLSASVGDRVTITCRASQGISSDLAWYQQKPGKAPKLLMYDASSLESGVPSRFSGSGSGTDFTLTISSLQPEDFATYYCQQFNSYPITFGQGTRLEIKR

SEQ ID NO: 15 - protein, 5 aa, Artificial Sequence
  39B9 VH CDR1
SYAIS

SEQ ID NO: 16 - protein, 17 aa, Artificial Sequence
  39B9 VH CDR2
GIIPLFDTTKYAQQFQG

SEQ ID NO: 17 - protein, 19 aa, Artificial Sequence
  39B9 VH CDR3
CARDRDILTDYYPMGGMDV

SEQ ID NO: 18 - protein, 5 aa, Artificial Sequence
  12A VH CDR1
NYDMY

SEQ ID NO: 19 - protein, 17 aa, Artificial Sequence
  12A VH CDR2
VILDDGNNNYYADSVKG

SEQ ID NO: 20 - protein, 13 aa, Artificial Sequence
  12A VH CDR3
CVRASPNWGLLDF

SEQ ID NO: 21 - protein, 5 aa, Artificial Sequence
  5C VH CDR1
SYDMY

SEQ ID NO: 22 - protein, 17 aa, Artificial Sequence
  5C VH CDR2
VILYDGNNKYYADSVKG

SEQ ID NO: 23 - protein, 13 aa, Artificial Sequence
  5C VH CDR3
CVRASPNWGLFDF

SEQ ID NO: 24 - protein, 11 aa, Artificial Sequence
  39B9 VL1 VL CDR1
RASQGISSVLA

SEQ ID NO: 25 - protein, 7 aa, Artificial Sequence
  IL-6 antibody VL CDR2
DASSLES

SEQ ID NO: 26 - protein, 9 aa, Artificial Sequence
  39B9 VL1 VL CDR3
QQSNSYPLT

SEQ ID NO: 27 - protein, 11 aa, Artificial Sequence
  39B9 VL5 VL CDR1
RASQDISSWLA

SEQ ID NO: 28 - protein, 11 aa, Artificial Sequence
  12A VL CDR1
RASQGISSWLA

SEQ ID NO: 29 - protein, 9 aa, Artificial Sequence
  12A VL CDR3
QQSNSYPIT

SEQ ID NO: 30 - protein, 11 aa, Artificial Sequence
  5C VL CDR1
RASQGISSVDA

SEQ ID NO: 31 - protein, 9 aa, Artificial Sequence
  VL CDR3 motif
  MISC_FEATURE (4)..(4): Xaa at position 4 is asparagine or glutamine
QQSXSYPLT

SEQ ID NO: 32 - protein, 17 aa, Artificial Sequence
  VH CDR2 motif
  MISC_FEATURE (5)..(5): Xaa at position 5 can be leucine or alanine
  MISC_FEATURE (7)..(7): Xaa at position 7 can be aspartic acid or glutamic acid
  MISC_FEATURE (14)..(14): Xaa at position 14 can be glutamine or lysine
GIIPXFXTTKYAQXFQG

SEQ ID NO: 33 - protein, 16 aa, Artificial Sequence
  VH CDR3 motif
  MISC_FEATURE (11)..(11): Xaa at position 11 is methionine or leucine
DRDILTDYYPXGGMDV

SEQ ID NO: 34 - protein, 8 aa, Artificial Sequence
  FRW3 mofitf
  MISC_FEATURE (4)..(4): Xaa at position 4 is phenylalanine or tyrosine
TAVXYCAR

SEQ ID NO: 35 - protein, 16 aa, Artificial Sequence
  NI-1201-WT VH CDR3
DRDILTDYYPMGGMDV

SEQ ID NO: 36 - protein, 8 aa, Artificial Sequence
  NI-1201-WT FRW3
TAVFYCAR

SEQ ID NO: 37 - protein, 17 aa, Artificial Sequence
  NI-1201-WT VH CDR2
GIIPLFDTTKYAQKFQG

SEQ ID NO: 38 - protein, 8 aa, Artificial Sequence [sequence omitted]

SEQ ID NO: 39 - protein, 16 aa, Artificial Sequence
  NI-1201-B VH CDR3
DRDILTDYYPLGGMDV

SEQ ID NO: 40 - protein, 17 aa, Artificial Sequence
  NI-1201-C VH CDR2
GIIPAFETTKYAQKFQG

SEQ ID NO: 41 - protein, 9 aa, Artificial Sequence
  NI-1201-D VL CDR3
QQSQSYPLT

SEQ ID NO: 42 - protein, 5 aa, Artificial Sequence
  anti-CD3 CDRH1
GYGMH

SEQ ID NO: 43 - protein, 17 aa, Artificial Sequence
  anti-CD3 CDRH2
VIWYDGSKKYYVDSVKG

SEQ ID NO: 44 - protein, 9 aa, Artificial Sequence
  anti-CD3 CDRH3
QMGYWHFDL

SEQ ID NO: 45 - protein, 11 aa, Artificial Sequence
  anti-CD3 CDRL1
RASQSVSSYLA

SEQ ID NO: 46 - protein, 7 aa, Artificial Sequence
  anti-CD3 CDRL2
DASNRAT

SEQ ID NO: 47 - protein, 10 aa, Artificial Sequence
  anti-CD3 CDRL3
QQRSNWPPLT

SEQ ID NO: 48 - protein, 118 aa, Artificial Sequence [sequence omitted]

SEQ ID NO: 49 - protein, 108 aa, Artificial Sequence [sequence omitted]

SEQ ID NO: 50 - protein, 448 aa, Artificial Sequence
  anti-CD3 HC
QVQLVESGGGVVQPGRSLRLSCAASGFKFSGYGMHWVRQAPGKGLEWVAVIWYDGSKKYYVDSVKGRFTISRDNSKNTLYLQMNSLRAEDTAVYYCARQMGYWHFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 51 - protein, 215 aa, Artificial Sequence
  anti-CD3 LC
EIVLTQSPATLSLSPGERATLSCRASQSVSSYLAWYQQKPGQAPRLLIYDASNRATGIPARFSGSGSGTDFTLTISSLEPEDFAVYYCQQRSNWPPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 52 - protein, 454 aa, Artificial Sequence
  TZLS-501 heavy chain
QVQLVQSGAEVKKPGSSVKVSCKASGGTFSSYAISWVRQAPGQGLEWMGGIIPLFDTTKYAQKFQGRVTITADESTSTAYMELSSLRSEDTAVYYCARDRDILTDYYPMGGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 53 - protein, 214 aa, Artificial Sequence
  TZLS-501 light chain
AIQLTQSPSSLSASVGDRVTITCRASQGISSVLAWYQQKPGKAPKLLIYDASSLESGVPSRFSGSGSGTDFTLTISSLQPEDFATYYCQQSNSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2023/0227555 A1
(43) Pub. Date: Jul. 20, 2023

(54) CD-3 ANTIBODIES FOR THE TREATMENT OF CORONAVIRUS

(71) Applicant: Tiziana Life Sciences PLC, London (GB)

(72) Inventors: Kunwar SHAILUBHAI, Line Lexington, PA (US); Howard L. Weiner, Brookline, MA (US)

(21) Appl. No.: 18/002,591

(22) PCT Filed: Jul. 30, 2021
(86) PCT No.: PCT/EP2021/071491

§ 371 Date: Dec. 20, 2022

Related U.S. Application Data
(60) Provisional application No. 63/058,978, filed on Jul. 30, 2020.

(51) Int. Cl. C07K 16/28; A61P 31/14

(52) U.S. Cl. CPC C07K 16/2809; A61P 31/14; C07K 2317/24; C07K 2317/565

(57) ABSTRACT

This invention provides methods for treating and preventing symptoms of coronavirus infections using antibodies that recognize CD3. The invention further provides routes of administration and formulations for said methods.

### CROSS REFERENCE TO RELATED APPLICATIONS

This application claims the benefit of priority to U.S. Provisional Patent Application Ser. No. 63/058,978 filed on Jul. 30, 2020, the contents of which are hereby incorporated by reference in their entireties.

### REFERENCE TO SEQUENCE LISTING

This application is being filed electronically via EFS-Web and includes an electronically submitted sequence listing in .txt format. The .txt file contains a sequence listing entitled “TIZI_028_001WO_SeqList_ST25.txt” created on Jul. 29, 2021 and having a size of 33 kilobytes. The sequence listing contained in this .txt file is part of the specification and is incorporated herein by reference in its entirety.

### FIELD OF THE INVENTION

The present invention relates to generally to composition and method of treating coronavirus infection and its variants, e.g., COVID-19, SARs, and MERS by administering an anti-CD3 antibody alone or in combination with a steroid, such as dexamethasone, or an anti-viral therapy.

### BACKGROUND

Coronaviruses are enveloped non-segmented positive sense RNA viruses belonging to the family Coronaviridae. Coronaviruses can cause multiple system infections mainly respiratory tract infections in humans, such as severe acute respiratory syndrome (SARS) and Middle East respiratory syndrome (MERS). A novel coronavirus (2019-nCoV, COVID-19) originating in Wuhan, China presents a respiratory viral pandemic to the world population. Current efforts are focused on containment and quarantine of infected individuals. This outbreak could be controlled with a protective vaccine to prevent COVID-19 infection but so far, no vaccine exists for treatment of this virus.

Human CD3 antigen consists of a minimum of four invariant polypeptide chains, which are non-covalently associated with the T-cell receptors on the surface of T-cells, and is generally now referred to as the CD3 antigen complex. It is intimately involved in the process of T-cell activation in response to antigen recognition by the T-cell receptors.

Due to the fundamental nature of CD3 in initiating an anti-antigen response, monoclonal antibodies against this receptor are capable of blocking or at least modulating the immune process and thus as useful as agents of disease

Accordingly, there exists a need for therapies that neutralize the biological activities CD3 to treat and prevent coronavirus infections such as COVID-19 and associated symptoms.

### SUMMARY

In one aspect, the disclosure provides a method of treating, preventing or alleviating a symptom of a coronavirus infection in a subject in need thereof comprising administering to the subject a composition comprising an anti-CD3 antibody. In some embodiments, the antibody is a monoclonal antibody. In some embodiments, the antibody is fully human or humanized.

In some embodiments, the anti-CD3 antibody comprises a heavy chain complementarity determining region 1 (CDRH1) comprising the amino acid sequence GYGMH (SEQ ID NO: 42), a heavy chain complementarity determining region 2 (CDRH2) comprising the amino acid sequence VIWYDGSKKYYVDSVKG (SEQ ID NO: 43), a heavy chain complementarity determining region 3 (CDRH3) comprising the amino acid sequence QMGYWHFDL (SEQ ID NO: 44), a light chain complementarity determining region 1 (CDRL1) comprising the amino acid sequence RASQSVSSYLA (SEQ ID NO: 45), a light chain complementarity determining region 2 (CDRL2) comprising the amino acid sequence DASNRAT (SEQ ID NO: 46), and a light chain complementarity determining region 3 (CDRL3) comprising the amino acid sequence QQRSNWPPLT (SEQ ID NO: 47). In some embodiments, the anti-CD3 antibody comprises a variable heavy chain amino acid sequence comprising the amino acid sequence of SEQ ID NO: 48 and a variable light chain amino acid sequence comprising the amino acid sequence of SEQ ID NO: 49. In some embodiments, the anti-CD3 antibody comprises a heavy chain amino acid sequence comprising the amino acid sequence of SEQ ID NO: 50 and a light chain amino acid sequence comprising the amino acid sequence of SEQ ID NO: 51.

In some embodiments, the coronavirus is SARS-CoV, SARS-CoV-2, MERS-CoV, or a mutant or a variant thereof. In some embodiments, the symptom of a coronavirus infection is one or more of hyperactive immune response, fever, gastrointestinal symptoms, respiratory symptoms, anosmia (loss of smell), dysgeusia (loss of taste), cough, headache, throat ache, pain when swallowing, dyspnea, difficult breathing, shortness of breath, nausea, vomiting, reduced 02 saturation, diarrhea, rhinorrhea, abdominal pain, myalgia, fever, conjunctivitis, and loss of appetite. In some embodiments, the hyperactive immune response comprises increased levels of at least one of interleukin 6 (IL-6), C-reactive protein (CRP), and D-dimer. In some embodiments, the levels of at least one of IL-6, CRP, and D-dimer are reduced.

In some embodiments, the subject has or is suspected of having a coronavirus infection. In some embodiments, the subject has been or thought to have been exposed to a coronavirus and has not yet developed symptoms of a coronavirus infection.

In some embodiments, the method further comprises administering to the subject a composition comprising dexamethasone.

In some embodiments, the composition is administered orally, mucosally, by inhalation, nasally, intravenously or any combination thereof. In some embodiments, the inhalation administration is by an inhaler or a nebulizer.

In some embodiments, the method further comprises administering an anti-TNFα antibody, an anti-CD20 antibody an anti-IFNγ antibody, an anti-Granulocyte-Macrophage Colony-Stimulating Factor antibody or an anti-IL-6R antibody. In some embodiments, the method further comprises administering to the subject an antiviral drug, an immune booster drug, vitamin C, Vitamin D, Vitamin E or any combination thereof. In some embodiments, the antiviral drug is Azidothymidine, Remdesivir or Actinomycin D.

In some embodiments, the anti-CD3 is administered nasally at a daily dose of 50 □g to 100 □g. In some embodiments, the daily dose is administered once daily. In some embodiments, the daily dose is administered for at least 10 consecutive days. In some embodiments, the anti-CD3 is administered orally at a daily dose of 1.0 to 2.5 mg.

In some embodiments, the subject is further administered dexamethasone. In some embodiments, the dexamethasone is administered by inhalation. In some embodiments, the administration by inhalation by a metered inhaler. In one aspect, the disclosure provides a nasal or inhalation formulation comprising an anti-CD3 antibody and dexamethasone.

In one aspect, the disclosure provides a method of treating, preventing, or alleviating a symptom of a disease or disorder in a subject in need thereof comprising administering to the subject a composition comprising an anti-CD3 antibody.

In some embodiments, the subject has an inflammatory disease or disorder. In some embodiments, the inflammatory disease or disorder is autoimmune encephalomyelitis, lupus, or arthritis. In some embodiments, the subject has a pulmonary disease or disorder. In some embodiments, the pulmonary disease or disorder is acute respiratory distress syndrome (ARDS). In some embodiments, the subject has a neurodegenerative disease. In some embodiments, the subject has a neurodegenerative disease or disorder. In some embodiments, the neurodegenerative disease or disorder is multiple sclerosis. In some embodiments, the multiple sclerosis is secondary progressive multiple sclerosis.

In one aspect, the disclosure provides a method of treating, preventing or alleviating a symptom of a disease or disorder in a subject in need thereof comprising: a) collecting a sample from the subject; b) measuring a marker for hyperactive immune response; and c) administering to the subject a composition comprising an anti-CD3 antibody based on a level of the marker. In some embodiments, the disease or disorder is a coronavirus infection, an inflammatory disease or disorder, a pulmonary disease or disorder, or a neurodegenerative disease or disorder. In some embodiments, the marker for hyperactive immune response is at least one of interleukin 6 (IL-6), C-reactive protein (CRP), D-dimer, interferon (IFN), interferon alpha (IFN-α), interferon gamma (IFN-γ), interleukin 1 beta 4-1(3), and/or CXCL10. In some embodiments, the level of the marker is elevated or high compared to a healthy subject. In some embodiments, the anti-CD3 antibody is foralumab.

Unless otherwise defined, all technical and scientific terms used herein have the same meaning as commonly understood by one of ordinary skill in the art to which this invention pertains. Although methods and materials similar or equivalent to those described herein can be used in the practice of the present invention, suitable methods and materials are described below. All publications, patent applications, patents, and other references mentioned herein are expressly incorporated by reference in their entirety. In cases of conflict, the present specification, including definitions, will control. In addition, the materials, methods, and examples described herein are illustrative only and are not intended to be limiting.

Other features and advantages of the invention will be apparent from and encompassed by the following detailed description and claims.

### BRIEF DESCRIPTION OF THE DRAWINGS

FIG. 1 is a diagram illustrating various stages of progression of COVID-19 pathogenesis. The progression of the diseases is anticipated to be retarded by using therapeutic intervention with combination of therapies as indicated. Treatment of dexamethasone is for suppression of underlying inflammatory mechanisms and treatment with anti-CD3 is to activate mucosal immune system to suppress cytokine storm.

FIG. 2 is an illustration showing the brief overviews of the clinical design. The patient population selected for the study would early stage patient who are symptomatic but have yet not progressed to severe stage requiring ventilator use. The clinical study consists of three arms randomized with 6 to 10 patients. The dose of dexamethasone administered using pMDI and Foralumab administered by nasal spray device are as indicated. The clinical endpoint and duration of the study are same as shown in the drawing.

FIG. 3 is an illustration showing the brief overviews of the clinical design. The patient population selected for the study would early stage patient who are symptomatic but have yet not progressed to severe stage requiring ventilator use. The clinical study consists of three arms randomized with 6 to 10 patients. The dose of azidothymidine administered and Foralumab administered by nasal spray device are as indicated. The clinical endpoint and duration of the study are same as shown in the drawing.

FIG. 4 are photographs of a) handheld metered dose inhaler (pMDI) and b) nasal spray device to be used for nasal administration of Foralumab. This device could be also used for nasal administration of anti-IL-6R.

FIG. 5 is a diagram showing a clinical study design of a method for treating COVID-19 with an anti-CD3 antibody (foralumab).

FIG. 6A shows a plot and chart demonstrating reduction of IL-6 in subjects suffering from COVID-19 treated with an anti-CD3 antibody (foralumab) and dexamethasone, anti-CD3 antibody alone (foralumab), or a placebo (control). IL=interleukin; CRP=C-reactive protein; Dexa=dexamethasone.

FIG. 6B shows a plot and chart demonstrating reduction of C-reactive protein in subjects suffering from COVID-19 treated with an anti-CD3 antibody (foralumab) and dexamethasone, anti-CD3 antibody (foralumab) alone, or a placebo (control). IL=interleukin; CRP=C-reactive protein; Dexa=dexamethasone.

FIG. 7 shows a series of lung CT scan images in subjects suffering from COVID-19 treated with an anti-CD3 antibody (foralumab) and dexamethasone (Dexa), anti-CD3 antibody (foralumab) alone, or a placebo (control).

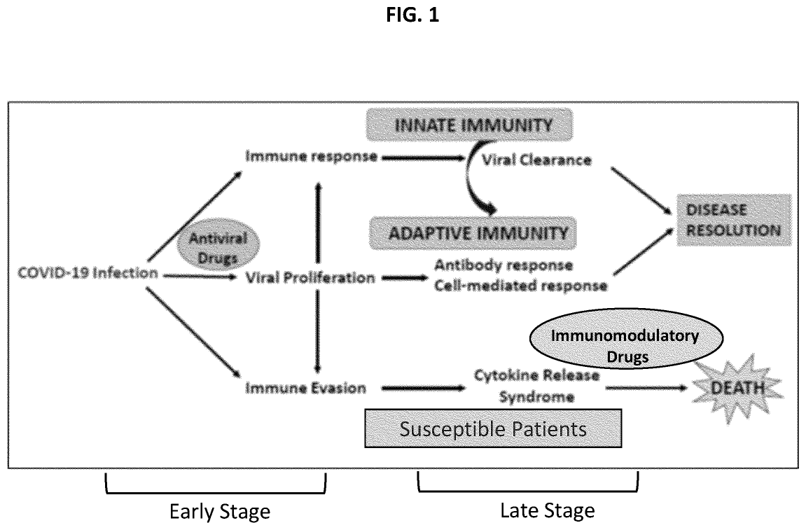

### DETAILED DESCRIPTION

The present invention provides monoclonal antibodies that specifically bind the human CD3 for the treatment, prevention or alleviating a symptom of a coronavirus infection such as SARS-CoV-2 (i.e., COVID-19), SARS-CoV-1, and Middle East respiratory syndrome coronavirus (MERS-CoV). These antibodies are collectively referred to herein as “huCD3” antibodies. The antibody can be e.g., a fully human antibody.

The exact mechanism of SARS-CoV-2 pathogenesis still remains to be discovered, it is suggested that the clinical manifestation of severe SARS-CoV-2 infection is also characterized by an over-exuberant immune response with lung lymphomononuclear cell infiltration and proliferation that may account for tissue damage more than the direct effect of viral replication.

SARS-CoV-2 involves a two-step process, a viral-mediated and an immune-mediated process. The viral mediated first step is characterized primarily by acute viral symptoms, fever, myalgias, cough, and is the direct result of the virus infecting the cells. During the viral phase, infection of cells in various organs via viral specific receptors occurs and is followed by clinical deterioration. The viral phase is followed by second phases that is characterized by an inappropriate exacerbated immune response which involves multiple cytokines and immune cells which induce an immune-mediated end organ damage The second immune mediated phase is associated with life threatening complications, most commonly—the Acute Respiratory Distress Syndrome (ARDS), which is the result from an exaggerated host response, and termed a Cytokine Storm.

Given the hyperactive inflammatory effects (i.e. hyperactive immune response) of coronaviral infections such as SARS-CoV-2 (COVID19), agents that modulate the immune response are being explored as adjunctive treatments for the management of moderate to critical COVID-19.

As disclosed herein, modulating regulatory T cells (Tregs) by administration of an anti-CD3 antibody can be a treatment directed at a hyperactive immune response. Tregs migrate into inflamed tissues, dampening inflammatory responses and hastening tissue repair. The inventors have conceived that, without being bound by theory, induction of regulatory T cells by administration of anti-CD3 antibodies can be used as a treatment in diseases and disorders associated with a hyperactive inflammatory effects, including, without limitation, COVID-19, ARDS, inflammatory disorders, and neurodegenerative diseases.

The inventors have shown that, surprisingly, the mucosal (oral and nasal) administration of an anti-CD3 monoclonal antibody is immunomodulatory and suppresses hyperactive immune responses in a large number of inflammatory and autoimmune disease including models of multiple sclerosis, diabetes, arthritis, lupus, colitis, and graft rejection. The mechanism of action includes the induction of regulatory T cells, downregulation of Th1 and Th17 cells, and downregulation of CD8 cells.

Furthermore, the inventors have shown in human studies where a fully humanized anti-CD3 Mab, foralumab was given nasally to healthy volunteers over a dose range of 10 ug, 50 ug, and 250 ug per dose, given on five consecutive days. Nasally administered foralumab at the 50 ug dose was well tolerated and suppressed cytotoxic CD8+ as well as perforin secreting CD8+ cells and the treatment also suppressed the pro-inflammatory cytokine IFN-γ. Thus, the oral or nasal administration of foralumab has immunomodulatory properties that would be beneficial to treat the hyperactive immune response that occurs with COVID infection.

Accordingly, in various aspects the invention provides method for treating immune activation in Covid-19 by administering anti-CD3 mAbs either alone or in combination with dexamethasone. The treatment may be administered a) anti-CD3 given nasally; b) anti-CD3 given orally; c) anti-CD3 given combination of nasal and oral administration and d) anti-CD3 given nasally or orally in combination with dexamethasone. Specifically, a suitable dose of anti-CD3 mAb may be administered nasally by hand-held spray device either alone or in combination with oral administration of anti-CD3. This invention also relates to direct lung delivery of anti-CD3 mAbs and dexamethasone, delivered by a hand-held inhaler for treatment of COVID-19 patients.

The direct delivery of drugs by inhalation is advantageous as same level of efficacy can be achieved at a much lower dose and side effects are also minimized as compared to other routes of administration. Inhalation therapy is efficient treatment of lung diseases due to direct drug delivery into the lung. Dry powder inhalers (DPIs) are portable solid powder delivery units without propellants. DPIs can directly target drugs into the deep sites of the lung. A metered dose inhaler (MDI) is a small device that delivers a measured amount of medication to your lungs. The calibrated dose of medication is delivered with each spray (puff) when patient breathe in.

### Anti-CD3 Antibodies

Antibodies specific for CD3 epsilon chain (CD3c) and antigen binding fragments thereof are referred to herein as an “anti-CD3 antibody” or “CD3 antibody”, and the compositions are referred to herein as an “anti-CD3 antibody compositions.” Any anti-CD3 antibody known in the art is suitable for use in the present disclosure. The anti-CD3 antibody is a monoclonal antibody.

The anti-CD3 antibodies can be any antibodies specific for CD3. The anti-CD3 antibody can be a polyclonal, monoclonal, recombinant, e.g., a chimeric, de-immunized or humanized, fully human, non-human, e.g., murine, single chain antibody or single domain antibody. In some embodiments the antibody has effector function and can fix complement. In some embodiments, the antibody has reduced or no ability to bind an Fc receptor. For example, the anti-CD3 antibody can be an isotype or subtype, fragment or other mutant, which does not support binding to an Fc receptor, e.g., it has a mutagenized or deleted Fc receptor binding region. The antibody can be coupled to a toxin or imaging agent.

A number of anti-CD3 antibodies are known, including but not limited to OKT3 (muromonab/Orthoclone OKT3. TM., Ortho Biotech, Raritan, N.J.; U.S. Pat. No. 4,361,549); hOKT3 (1 (Herold et al., N. E. J. M. 346(22): 1692-1698 (2002); HuM291 (Nuvion. TM., Protein Design Labs, Fremont, Calif.); gOKT3-5 (Alegre et al., J. Immunol. 148(11):3461-8 (1992); 1F4 (Tanaka et al., J. Immunol. 142:2791-2795 (1989)); G4.18 (Nicolls et al., Transplantation 55:459-468 (1993)); 145-2C11 (Davignon et al., J. Immunol. 141(6):1848-54 (1988)); and as described in Frenken et al., Transplantation 51(4):881-7 (1991); U.S. Pat. Nos. 6,491,9116, 6,406,696, and 6,143,297).

Methods for making such antibodies are also known. A full-length CD3 protein or antigenic peptide fragment of CD3 can be used as an immunogen, or can be used to identify anti-CD3 antibodies made with other immunogens, e.g., cells, membrane preparations, and the like, e.g., E rosette positive purified normal human peripheral T cells, as described in U.S. Pat. Nos. 4,361,549 and 4,654,210. The anti-CD3 antibody can bind an epitope on any domain or region on CD3.

Chimeric, humanized, de-immunized, or completely human antibodies are desirable for applications which include repeated administration, e.g., therapeutic treatment of human subjects.

Chimeric antibodies contain portions of two different antibodies, typically of two different species. Generally, such antibodies contain human constant regions and variable regions from another species, e.g., murine variable regions. For example, mouse/human chimeric antibodies have been reported which exhibit binding characteristics of the parental mouse antibody, and effector functions associated with the human constant region. See, e.g., Cabilly et al., U.S. Pat. No. 4,816,567; Shoemaker et al., U.S. Pat. No. 4,978,745; Beavers et al., U.S. Pat. No. 4,975,369; and Boss et al., U.S. Pat. No. 4,816,397, all of which are incorporated by reference herein. Generally, these chimeric antibodies are constructed by preparing a genomic gene library from DNA extracted from pre-existing murine hybridomas (Nishimura et al., Cancer Research, 47:999 (1987)). The library is then screened for variable region genes from both heavy and light chains exhibiting the correct antibody fragment rearrangement patterns. Alternatively, cDNA libraries are prepared from RNA extracted from the hybridomas and screened, or the variable regions are obtained by polymerase chain reaction. The cloned variable region genes are then ligated into an expression vector containing cloned cassettes of the appropriate heavy or light chain human constant region gene. The chimeric genes can then be expressed in a cell line of choice, e.g., a murine myeloma line. Such chimeric antibodies have been used in human therapy.

Humanized antibodies are known in the art. Typically, “humanization” results in an antibody that is less immunogenic, with complete retention of the antigen-binding properties of the original molecule. In order to retain all the antigen-binding properties of the original antibody, the structure of its combining-site has to be faithfully reproduced in the “humanized” version. This can potentially be achieved by transplanting the combining site of the nonhuman antibody onto a human framework, either (a) by grafting the entire nonhuman variable domains onto human constant regions to generate a chimeric antibody (Morrison et al., Proc. Natl. Acad. Sci., USA 81:6801 (1984); Morrison and Oi, Adv. Immunol. 44:65 (1988) (which preserves the ligand-binding properties, but which also retains the immunogenicity of the nonhuman variable domains); (b) by grafting only the nonhuman CDRs onto human framework and constant regions with or without retention of critical framework residues (Jones et al. Nature, 321:522 (1986); Verhoeyen et al., Science 239:1539 (1988)); or (c) by transplanting the entire nonhuman variable domains (to preserve ligand-binding properties) but also “cloaking” them with a human-like surface through judicious replacement of exposed residues (to reduce antigenicity) (Padlan, Molec. Immunol. 28:489 (1991)).

Humanization by CDR grafting typically involves transplanting only the CDRs onto human fragment onto human framework and constant regions. Theoretically, this should substantially eliminate immunogenicity (except if allotypic or idiotypic differences exist). However, it has been reported that some framework residues of the original antibody also need to be preserved (Riechmann et al., Nature 332:323 (1988); Queen et al., Proc. Natl. Acad. Sci. USA 86:10,029 (1989)). The framework residues which need to be preserved can be identified by computer modeling. Alternatively, critical framework residues may potentially be identified by comparing known antibody combining site structures (Padlan, Molec. Immun. 31(3):169-217 (1994)). The compositions and methods of the disclosure also include partially humanized antibodies, in which the 6 CDRs of the heavy and light chains and a limited number of structural amino acids of the murine monoclonal antibody are grafted by recombinant technology to the CDR-depleted human IgG scaffold (Jones et al., Nature 321:522-525 (1986)).

Deimmunized antibodies are made by replacing immunogenic epitopes in the murine variable domains with benign amino acid sequences, resulting in a deimmunized variable domain. The deimmunized variable domains are linked genetically to human IgG constant domains to yield a deimmunized antibody (Biovation, Aberdeen, Scotland).

The anti-CD3 antibody can also be a single chain antibody. A single-chain antibody (scFV) can be engineered (see, for example, Colcher et al., Ann. N. Y. Acad. Sci. 880:263-80 (1999); and Reiter, Clin. Cancer Res. 2:245-52 (1996)). The single chain antibody can be dimerized or multimerized to generate multivalent antibodies having specificities for different epitopes of the same target CD3 protein. In some embodiments, the antibody is monovalent, e.g., as described in Abbs et al., Ther. Immunol. 1(6):325-31 (1994), incorporated herein by reference.

Exemplary anti-CD3 antibodies, comprise a heavy chain complementarity determining region 1 (CDRH1) comprising the amino acid sequence GYGMH (SEQ ID NO: 42), a heavy chain complementarity determining region 2 (CDRH2) comprising the amino acid sequence VIWYDGSKKYYVDSVKG (SEQ ID NO: 43), a heavy chain complementarity determining region 3 (CDRH3) comprising the amino acid sequence QMGYWHFDL (SEQ ID NO: 44), a light chain complementarity determining region 1 (CDRL1) comprising the amino acid sequence RASQSVSSYLA (SEQ ID NO: 45), a light chain complementarity determining region 2 (CDRL2) comprising the amino acid sequence DASNRAT (SEQ ID NO: 46), and a light chain complementarity determining region 3 (CDRL3) comprising the amino acid sequence QQRSNWPPLT (SEQ ID NO: 47).

In some embodiments, the anti-CD3 antibody comprises a variable heavy chain amino acid sequence comprising QVQLVESGGGVVQPGRSLRLSCAASGFKFSGYGMHWVRQAPGKGLEWVAVIWYDGS KKYYVDSVKGRFTISRDNSKNTLYLQMNSLRAEDTAVYYCARQMGYWHFDLWGRGT LVTVSS (SEQ ID NO: 48) and a variable light chain amino acid sequence comprising EIVLTQSPATLSLSPGERATLSCRASQSVSSYLAWYQQKPGQAPRLLIYDASNRATGIPA RFSGSGSGTDFTLTISSLEPEDFAVYYCQQRSNWPPLTFGGGTKVEIK (SEQ ID NO: 49).

Preferably, the anti-CD3 antibody comprises a heavy chain amino acid sequence comprising: QVQLVESGGGVVQPGRSLRLSCAASGFKFSGYGMHWVRQAPGKGLEWVAVIWYDGS KKYYVDSVKGRFTISRDNSKNTLYLQMNSLRAEDTAVYYCARQMGYWHFDLWGRGT LVTVSSASTKGPSVFPLAPSSKSTSGGTAALGCLVKDYFPEPVTVSWNSGALTSGVHTFP AVLQSSGLYSLSSVVTVPSSSLGTQTYICNVNHKPSNTKVDKRVEPKSCDKTHTCPPCPA PEAEGGPSVFLFPPKPKDTLMISRTPEVTCVVVDVSHEDPEVKFNWYVDGVEVHNAKT KPREEQYNSTYRVVSVLTVLHQDWLNGKEYKCKVSNKALPAPIEKTISKAKGQPREPQ VYTLPPSREEMTKNQVSLTCLVKGFYPSDIAVEWESNGQPENNYKTTPPVLDSDGSFFL YSKLTVDKSRWQQGNVFSCSVMHEALHNHYTQKSLSLSPGK (SEQ ID NO: 50) and a light chain amino acid sequence comprising: EIVLTQSPATLSLSPGERATLSCRASQSVSSYLAWYQQKPGQAPRLLIYDASNRATGIPA RFSGSGSGTDFTLTISSLEPEDFAVYYCQQRSNWPPLTFGGGTKVEIKRTVAAPSVFIFPPS DEQLKSGTASVVCLLNNFYPREAKVQWKVDNALQSGNSQESVTEQDSKDSTYSLSSTL TLSKADYEKHKVYACEVTHQGLSSPVTKSFNRGEC (SEQ ID NO: 51). This anti-CD3 antibody is referred to herein as NI-0401, Foralumab, or 28F11-AE. (See e.g., Dean Y, Dépis F, Kosco-Vilbois M. “Combination therapies in the context of anti-CD3 antibodies for the treatment of autoimmune diseases.” Swiss Med Wkly. (2012) (the contents of which are hereby incorporated by reference in its entirety).

In some embodiments, the anti-CD3 antibody is a fully human antibody or a humanized antibody. In some embodiments, the anti-CD3 antibody formulation includes a full length anti-CD3 antibody. In some embodiments, the anti-CD3 antibody formulation includes an antibody fragment that specifically binds CD3. In some embodiments, the anti-CD3 antibody formulation includes a combination of full-length anti-CD3 antibodies and antigen binding fragments that specifically bind CD3.

In some embodiments, the antibody or antigen-binding fragment thereof that binds CD3 is a monoclonal antibody, domain antibody, single chain, Fab fragment, a F(ab′)2 fragment, a scFv, a scAb, a dAb, a single domain heavy chain antibody, or a single domain light chain antibody. In some embodiments, such an antibody or antigen-binding fragment thereof that binds CD3 is a mouse, other rodent, chimeric, humanized or fully human monoclonal antibody.

Optionally, the anti-CD3 antibody or antigen binding fragment thereof used in the formulations of the disclosure includes at least one amino acid mutation. Typically, the mutation is in the constant region. The mutation results in an antibody that has an altered effector function. An effector function of an antibody is altered by altering, i.e., enhancing or reducing, the affinity of the antibody for an effector molecule such as an Fc receptor or a complement component. For example, the mutation results in an antibody that is capable of reducing cytokine release from a T-cell. For example, the mutation is in the heavy chain at amino acid residue 234, 235, 265, or 297 or combinations thereof. Preferably, the mutation results in an alanine residue at either position 234, 235, 265 or 297, or a glutamate residue at position 235, or a combination thereof.

Preferably, the anti-CD3 antibody provided herein contains one or more mutations that prevent heavy chain constant region-mediated release of one or more cytokine(s) in vivo.

In some embodiments, the anti-CD3 antibody or antigen binding fragment thereof used in the formulations of the disclosure is a fully human antibody. The fully human CD3 antibodies used herein include, for example, a L234A and L235E mutation in the Fc region, such that cytokine release upon exposure to the anti-CD3 antibody is significantly reduced or eliminated. The L234A and L235E mutation in the Fc region of the anti-CD3 antibodies provided herein reduces or eliminates cytokine release when the anti-CD3 antibodies are exposed to human leukocytes, whereas the mutations described below maintain significant cytokine release capacity. For example, a significant reduction in cytokine release is defined by comparing the release of cytokines upon exposure to the anti-CD3 antibody having an L234A and L235E mutation in the Fc region to level of cytokine release upon exposure to another anti-CD3 antibody having one or more of the mutations described below. Other mutations in the Fc region include, for example, L234A and L235A, L235E, N297A, D265A, or combinations thereof.

The term “cytokine” refers to all human cytokines known within the art that bind extracellular receptors expressed on the cell surface and thereby modulate cell function, including but not limited to IL-2, IFN-gamma, TNF-α, IL-4, IL-5, IL-6, IL-9, IL-10, and IL-13.

The anti-CD3 formulation comprises a unit dose of the anti-CD3 antibody in the range of: about 0.01 mg to about 25 mg; or 0.01 mg to about 10 mg. For example, the unit dose is about 0.01, 0.02, 0.03, 0.04, 0.50, 0.06, 0.07, 0.08, 0.09, 0.1, 0.2, 0.3, 0.4, 0.5, 0.6, 0.7, 0.8, 0.9, 1.0, 1.5, 2.0, 2.5, 3.0, 3.5, 4.0, 5.0, 5.5, 6.0, 6.5, 7.0, 7.5, 8.0, 8.5, 9, 9.5, 10 mg or more. Preferably, the unit dose is 0.05 mg, 0.1 mg, 0.5 mg, 1.0 mg, 2.5 mg, 5.0 mg or 10 mg.

The anti-CD3 antibody formulation includes one or more salts (a buffering salt), one or more polyols and one or more excipients. The formulations of the present disclosure may also contain buffering agents, or preservatives. The anti-CD3 antibody formulation is buffered in a solution at a pH in the range of about 4 to 8; in the range of about 4 to 7; in the range of about 4 to 6; in the range of about 5 to 6; or in the range of about 5.5 to 6.5. Preferably, the pH is 5.5.

Examples of salts include those prepared from the following acids: hydrochloric, hydrobromic, sulfuric, nitric, phosphoric, maleic, acetic, salicylic, citric, boric, formic, malonic, succinic, and the like. Such salts can also be prepared as alkaline metal or alkaline earth salts, such as sodium, potassium or calcium salts. Examples of buffering agents include phosphate, citrate, acetate, and 2-(N-morpholino)ethanesulfonic acid (MES).

The formulations of the present disclosure may include a buffer system. As used in this application, the terms “buffer” or “buffer system” is meant a compound that, usually in combination with at least one other compound, provides a buffering system in solution that exhibits buffering capacity, that is, the capacity to neutralize, within limits, either acids or bases (alkali) with relatively little or no change in the original pH.

Buffers include borate buffers, phosphate buffers, calcium buffers, and combinations and mixtures thereof. Borate buffers include, for example, boric acid and its salts, for example, sodium borate or potassium borate. Borate buffers also include compounds such as potassium tetraborate or potassium metaborate that produce borate acid or its salt in solutions.

A phosphate buffer system includes one or more monobasic phosphates, dibasic phosphates and the like. Particularly useful phosphate buffers are those selected from phosphate salts of alkali and/or alkaline earth metals. Examples of suitable phosphate buffers include one or more of sodium dibasic phosphate (Na2HPO4), sodium monobasic phosphate (NaH2PO4) and potassium monobasic phosphate (KH2PO4). The phosphate buffer components frequently are used in amounts from 0.01% or to 0.5% (w/v), calculated as phosphate ion.

Other known buffer compounds can optionally be added to the according to the CD3 formulations, for example, citrates, sodium bicarbonate, TRIS, and the like. Other ingredients in the solution, while having other functions, may also affect the buffer capacity. For example, EDTA, often used as a complexing agent, can have a noticeable effect on the buffer capacity of a solution.

Preferred salts for use in the formulation of the disclosure include sodium chloride, sodium acetate, sodium acetate trihydrate and sodium citrate.

The concentration of salt in the formulations according to the disclosure is between about 10 mM and 500 mM, between about 25 m and 250 mM, between about 25 nM and 150 mM.

The sodium acetate trihydrate is at a concentration in the range of about 10 mM to 100 mM. For example, the sodium acetate trihydrate is at about 10, 15, 20, 25, 30, 35, 40, 45, 50, 55, 60, 65, 70, 75, 80, 85, 90, 95 or 100 mM. Preferably, the sodium acetate trihydrate is at 25 mM.

The sodium chloride at a concentration in the range of about 50 mM to 500 mM. For example, the sodium chloride is at about 50, 55, 60, 65, 70, 75, 80, 85, 90, 95, 100. 125, 150, 175, 200, 225, 250, 275, 300, 325, 350, 375, 400, 425, 450, 475 or 500 mM. Preferably, the sodium chloride is at a concentration of about 125 mM.

The sodium citrate is at a concentration in the range of about 10 mM to 100 mM For example the sodium citrate is at about 10, 15, 20, 25, 30, 35, 40, 45, 50, 55, 60, 65, 70, 75, 80, 85, 90, 95 or 100 mM. Preferably, the sodium citrate is in the range of about 25 to 50 mM.

In some embodiments, the salt is sodium acetate trihydrate at a concentration in the range of about 25 mm to 100 mm and sodium chloride at a concentration in the range of about 150 mm to 500 mm.

Preferably, the formulation includes about 25 mM sodium acetate trihydrate and about 150 mM sodium chloride.

The formulation includes one or more polyols as a bulking agent and/or stabilizing excipients. Polyols include for example, trehalose, mannitol, maltose, lactose, sucrose, sorbitol, or glycerol. The polyols is at a concentration in the range of about 0.1% to 50% or 5% to 25%. For example, the polyol is at about 1, 2, 3, 4, 5, 10, 15, 20, 25, 30, 35, 40, 45 or 50%.

In some embodiments, the polyol is trehalose at a concentration in the range of about 1% to 50% or 5% to 25%. For example, the trehalose is at about 1, 2, 3, 4, 5, 10, 15, 20, 25, 30, 35, 40, 45 or 50%. Preferably the trehalose is at a concentration of about 10% or about 20%. Most preferably, the trehalose is at a concentration of about 20%.

In some embodiments, the polyol is sorbitol at a concentration in the range of about 1% to about 10%. In some embodiments, the polyol is glycerol at a concentration in the range of about 1% to about 10%.

In some embodiments, the polyol is mannitol at a concentration in the range of about 0.1% to about 10%. In some embodiments, the polyol is maltose at a concentration in the range of about 1% to about 10%.

The formulation includes one or more excipients and/or surfactants to suppress or otherwise reduce antibody aggregation. Suitable excipients to reduce antibody aggregation include, by way of non-limiting example, a surfactant such as, by way of non-limiting example, Polysorbate 20 or Polysorbate 80. In some embodiments, the Polysorbate 20 or Polysorbate 80 is present at a concentration in the range of about 0.01 to 1% or about 0.01 to 0.05%. For example the Polysorbate 20 or Polysorbate 80 is at a concentration of about 0.01. 0.02, 0.03, 0.04, 0.05, 0.06, 0.07. 0.08, 0.09, 0.1, 0.2, 0.3. 0.4, 0.5, 0.6, 0.7, 0.8. 0.9, or 1.0%.

Preferably the surfactant is Polysorbate 80 at a concentration in the range of about 0.01 to 0.05%. More preferably, the Polysorbate 80 is at 0.02%.

The formulation includes one or more excipients to reduce antibody oxidation. Suitable excipients to reduce antibody oxidation include, by way of non-limiting example, antioxidants. Antioxidants include for example, methionine, D-arginine, BHT or ascorbic acid. The antioxidant is present at a concentration in the range of about 0.01% to 1%; 0.1% to 1%; or 0.1% to 0.5%. In some embodiments, the antioxidant is methionine. In some embodiments, the methionine is present at a concentration in the range of about 0.01% to 1%; 0.1% to 1%; or 0.1% to 0.5%. For example, the methionine is present at a concentration of about 0.01. 0.02, 0.03, 0.04, 0.05, 0.06, 0.07. 0.08, 0.09, 0.1, 0.2, 0.3. 0.4, 0.5, 0.6, 0.7, 0.8. 0.9, or 1.0%. Preferably, the methionine is at about 0.1%.

The formulation includes one or more chelating agents, such as for example ethylenediaminetetraacetic acid (EDTA). The chelating agent is at a concentration in the range of 0.01% to 1%; 0.1% to 1%; or 0.1% to 0.5%. For example, the chelating agent is present at a concentration of about 0.01. 0.02, 0.03, 0.04, 0.05, 0.06, 0.07. 0.08, 0.09, 0.1, 0.2, 0.3. 0.4, 0.5, 0.6, 0.7, 0.8. 0.9, or 1.0%. Preferably, the chelating agent is EDTA at a concentration of about 0.1%.

In some embodiments, the formulation includes one or more excipients to increase stability. In some embodiments, the excipient to increase stability is human serum albumin. In some embodiments, the human serum albumin is present in the range of about 1 mg to about 5 mg.

In some embodiments, the formulation includes magnesium stearate (Mg stearate), an amino acid, or both mg-stearate and an amino acid. Suitable amino acids include for example, leucine, arginine, histidine, or combinations thereof.

In some embodiments the one or more additional excipients is low moisture microcrystalline cellulose, such as Avicel, polyethylene glycols (PEG), or a starch.

Further examples of pharmaceutically acceptable carriers and excipients useful for the formulations of the present disclosure include, but are not limited to binders, fillers, disintegrants, lubricants, anti-microbial agents, antioxidant, and coating agents such as: BINDERS: corn starch, potato starch, other starches, gelatin, natural and synthetic gums such as acacia, xanthan, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone (e.g., povidone, crospovidone, copovidone, etc.), methyl cellulose, Methocel, pre-gelatinized starch (e.g., STARCH 1500® and STARCH 1500 LM®, sold by Colorcon, Ltd.), hydroxypropyl methyl cellulose, microcrystalline cellulose (FMC Corporation, Marcus Hook, Pa., USA), Emdex, Plasdone, or mixtures thereof, FILLERS: talc, calcium carbonate (e.g., granules or powder), dibasic calcium phosphate, tribasic calcium phosphate, calcium sulfate (e.g., granules or powder), microcrystalline cellulose, powdered cellulose, dextrates, kaolin, mannitol, silicic acid, sorbitol, starch, pre-gelatinized starch, dextrose, fructose, honey, lactose anhydrate, lactose monohydrate, lactose and aspartame, lactose and cellulose, lactose and microcrystalline cellulose, maltodextrin, maltose, mannitol, microcrystalline cellulose &amp; guar gum, molasses, sucrose, or mixtures thereof, DISINTEGRANTS: agar-agar, alginic acid, calcium carbonate, microcrystalline cellulose, croscarmellose sodium, crospovidone, polacrilin potassium, sodium starch glycolate, (such as Explotab), potato or tapioca starch, other starches, pre-gelatinized starch, clays, other algins, other celluloses, gums (like gellan), low-substituted hydroxypropyl cellulose, ployplasdone, or mixtures thereof, LUBRICANTS: calcium stearate, magnesium stearate, mineral oil, light mineral oil, glycerin, sorbitol, mannitol, polyethylene glycol, other glycols, compritol, stearic acid, sodium lauryl sulfate, sodium stearyl fumarate, (such as Pruv), vegetable based fatty acids lubricant, talc, hydrogenated vegetable oil (e.g., peanut oil, cottonseed oil, sunflower oil, sesame oil, olive oil, corn oil and soybean oil), zinc stearate, ethyl oleate, ethyl laurate, agar, syloid silica gel (AEROSIL 200, W.R. Grace Co., Baltimore, Md. USA), a coagulated aerosol of synthetic silica (Deaussa Co., Piano, TX USA), a pyrogenic silicon dioxide (CAB-O-SIL, Cabot Co., Boston, Mass. USA), or mixtures thereof, ANTI-CAKING AGENTS: calcium silicate, magnesium silicate, silicon dioxide, colloidal silicon dioxide, talc, or mixtures thereof, ANTIMICROBIAL AGENTS: benzalkonium chloride, benzethonium chloride, benzoic acid, benzyl alcohol, butyl paraben, cetylpyridinium chloride, cresol, chlorobutanol, dehydroacetic acid, ethylparaben, methylparaben, phenol, phenylethyl alcohol, phenoxyethanol, phenylmercuric acetate, phenylmercuric nitrate, potassium sorbate, propylparaben, sodium benzoate, sodium dehydroacetate, sodium propionate, sorbic acid, thimersol, thymo, or mixtures thereof, ANTIOXIDANTS: ascorbic acid, BHA, BHT, EDTA, or mixture thereof, and COATING AGENTS: sodium carboxymethyl cellulose, cellulose acetate phthalate, ethylcellulose, gelatin, pharmaceutical glaze, hydroxypropyl cellulose, hydroxypropyl methylcellulose (hypromellose), hydroxypropyl methyl cellulose phthalate, methylcellulose, polyethylene glycol, polyvinyl acetate phthalate, shellac, sucrose, titanium dioxide, carnauba wax, microcrystalline wax, gellan gum, maltodextrin, methacrylates, microcrystalline cellulose and carrageenan or mixtures thereof.

The formulation can also include other excipients and categories thereof including but not limited to Pluronic®, Poloxamers (such as Lutrol® and Poloxamer 188), ascorbic acid, glutathione, protease inhibitors (e.g. soybean trypsin inhibitor, organic acids), pH lowering agents, creams and lotions (like maltodextrin and carrageenans); materials for chewable tablets (like dextrose, fructose, lactose monohydrate, lactose and aspartame, lactose and cellulose, maltodextrin, maltose, mannitol, microcrystalline cellulose and guar gum, sorbitol crystalline); parenterals (like mannitol and povidone); plasticizers (like dibutyl sebacate, plasticizers for coatings, polyvinylacetate phthalate); powder lubricants (like glyceryl behenate); soft gelatin capsules (like sorbitol special solution); spheres for coating (like sugar spheres); spheronization agents (like glyceryl behenate and microcrystalline cellulose); suspending/gelling agents (like carrageenan, gellan gum, mannitol, microcrystalline cellulose, povidone, sodium starch glycolate, xanthan gum); sweeteners (like aspartame, aspartame and lactose, dextrose, fructose, honey, maltodextrin, maltose, mannitol, molasses, sorbitol crystalline, sorbitol special solution, sucrose); wet granulation agents (like calcium carbonate, lactose anhydrous, lactose monohydrate, maltodextrin, mannitol, microcrystalline cellulose, povidone, starch), caramel, carboxymethylcellulose sodium, cherry cream flavor and cherry flavor, citric acid anhydrous, citric acid, confectioner's sugar, D&C Red No. 33, D&C Yellow #10 Aluminum Lake, disodium edetate, ethyl alcohol 15%, FD&C Yellow No. 6 aluminum lake, FD&C Blue #1 Aluminum Lake, FD&C Blue No. 1, FD&C blue no. 2 aluminum lake, FD&C Green No. 3, FD&C Red No. 40, FD&C Yellow No. 6 Aluminum Lake, FD&C Yellow No. 6, FD&C Yellow No. 10, glycerol palmitostearate, glyceryl monostearate, indigo carmine, lecithin, manitol, methyl and propyl parabens, mono ammonium glycyrrhizinate, natural and artificial orange flavor, pharmaceutical glaze, poloxamer 188, Polydextrose, polysorbate 20, polysorbate 80, polyvidone, pregelatinized corn starch, pregelatinized starch, red iron oxide, saccharin sodium, sodium carboxymethyl ether, sodium chloride, sodium citrate, sodium phosphate, strawberry flavor, synthetic black iron oxide, synthetic red iron oxide, titanium dioxide, and white wax.

The CD3 antibodies formulated for enteral, parenteral, or nasal administration. For example, the CD3 antibodies are formulated for nasal, oral, inhalation, subcutaneous or intravenous administration.

For enteral administration, i.e., oral, the formulations may be a capsule or a tablet. Parental administration includes intravenous, subcutaneous, intramuscular, and intra-articular administration and may be a liquid or lyophilized powder in a sealed vial or other container. Preferred oral dose ranges is 0.1 mg to 5 mg daily. For example, a dose 0.1 mg, 0.2 mg, 0.3 mg, 0.4 mg, 0.5 mg, 0.6 mg, 0.7 mg, 0.8 mg, 0.9 mg, 1.0 mg, 1.5 mg, 2.0 mg, 2.5 mg, 3.0 mg, 3.5 mg, 4.0 mg, or 5.0 mg is administered daily. Administration of the dose is once daily or twice daily.

For nasal administration, the formulations may be an aerosol in a sealed vial or other suitable container. Preferred nasal dose ranges is 0.05 mg to 1 mg daily. For example, a dose 0.05 mg, 0.06 mg, 0.07 mg, 0.08 mg, 0.09 mg, 0.1 mg, 0.2 mg, 0.3 mg, 0.4 mg, 0.5 mg, 0.6 mg, 0.7 mg, 0.8 mg, 0.9 mg, or 1.0 mg, is administered daily. The does is equally split between each nostril. Administration of the dose is once daily or twice daily.

In some embodiments, the anti-CD3 antibody formulation is a subcutaneous formulation. In some embodiments, the subcutaneous anti-CD3 antibody formulation is housed in a sealed vial or other container. Preferred subcutaneous dose ranges is 0.2 mg to 5 mg daily. For example, a dose 0.2 mg, 0.3 mg, 0.4 mg, 0.5 mg, 0.6 mg, 0.7 mg, 0.8 mg, 0.9 mg, 1.0 mg, 1.5 mg, 2.0 mg, 2.5 mg, 3.0 mg, 3.5 mg, 4.0 mg, or 5.0 mg is administered daily. Administration of the dose is once daily or twice daily. A preferred formulation for subcutaneous administration is a preferred dosage of anti-CD3 antibody in 25 mM sodium acetate buffer, 125 mM sodium chloride with 0.02% polysorbate 80, at pH 5.5.

In some embodiments, the anti-CD3 antibody formulation is inhalation formulation. For inhalation administration, the formulations may be an aerosol in a sealed vial or other suitable container. Administration by inhalation may be in the form of an inhaler or a nebulizer. The nebulizer and/or inhaler is handheld. Optionally, the nebulizer and/or inhaler can be of different sizes to fit children and/or adults.

Preferred inhalation dose ranges is 0.1 mg to 5 mg daily. For example, a dose 0.1 mg, 0.2 mg, 0.3 mg, 0.4 mg, 0.5 mg, 0.6 mg, 0.7 mg, 0.8 mg, 0.9 mg, 1.0 mg, 1.5 mg, 2.0 mg, 2.5 mg, 3.0 mg, 3.5 mg, 4.0 mg, or 5.0 mg is administered daily. Administration of the dose is once daily or twice daily.

Particles of a particle formulation have diameters of between about 1 mm to about 5 mm, e.g., less than 5 mm in diameter, less than 4 mm in diameter, less than 3 mm in diameter, less than 2 mm in diameter, and about 1 mm in diameter.

Particles of a particle formulation comprising an anti-CD3 antibody or antigen-binding fragment thereof have average diameters of between about 0.1 mm to about 50 mm. Particles of a particle formulation comprising an anti-CD3 antibody or antigen-binding fragment thereof have average diameters of between about 1 mm to about 10 mm, e.g., less than 10 mm in average diameter, less than 9 mm in average diameter, less than 8 mm in average diameter, less than 7 mm in average diameter, less than 6 mm in average diameter, less than 5 mm in average diameter, less than 4 mm in average diameter, less than 3 mm in average diameter, and about 2 mm in average diameter. In some embodiments, particles have average diameters of between about 2 mm and 5 mm. In some embodiments, the particles have an average diameter between 2 mm and 5 mm, where each particle is less than about 50 mm in diameter.

In some embodiments the CD3 antibody is an extended and controlled release formulation. Methods of producing extended and controlled release formulation are known in the art and includes for example the use or macroporous beads.

In some embodiments, the anti-CD3 antibody formulation includes a full length anti-CD3 antibody. In some embodiments, the anti-CD3 antibody formulation includes an antibody fragment that specifically binds CD3. In some embodiments, the anti-CD3 antibody formulation includes a combination of full-length anti-CD3 antibodies and antigen binding fragments that specifically bind CD3.

### Methods of Treatment, Prevention, and Alleviating Symptoms

The disclosure provides methods of treating, preventing, or alleviating a symptom of a coronavirus infection, cytokine release syndrome (Shimabukuro-Vornhagen et al. J Immunother Cancer 6(1):56 (2018), herein incorporated by reference), inflammatory disease, acute respiratory distress syndrome (ARDS), neurodegenerative, or a pulmonary disease in a subject (i.e. a patient) in need thereof comprising administering to the subject a composition comprising an CD3 antibody (anti-CD3).

The disclosure further provides methods of treating, preventing, or alleviating a symptom of coronavirus infection, inflammatory disease, acute respiratory distress syndrome (ARDS), neurodegenerative, or a pulmonary disease in a subject in need thereof comprising administering to the subject: a composition comprising a CD3 antibody and a composition comprising dexamethasone, wherein administration of the composition comprising an CD3 antibody and administration of the composition comprising dexamethasone can occur in any order or simultaneously.

The CD3 antibodies described herein may be used as therapeutic agents. Such agents will generally be employed to treat, alleviate, and/or prevent a disease or pathology associated with a coronavirus infection, inflammatory disease, acute respiratory distress syndrome (ARDS), cytokine release syndrome, neurodegenerative, or a pulmonary disease in a subject. A therapeutic regimen is carried out by identifying a subject, e.g., a human patient suffering from (or at risk of developing) diseases and disorders described herein using standard methods. An CD3 antibody preparation, preferably one having high specificity and high affinity for its target antigen, is administered to the subject and will generally have an effect due to its binding with the target.

Administration of the antibody abrogates or inhibits, interferes with the signaling, or otherwise modulates the function of the target (e.g., CD3) and can modulate cellular behavior. Without being bound by theory, administration of an anti-CD3 antibody can, for example, induce T regulatory cells (Tregs) and suppress inflammation in models of autoimmunity. In some embodiments, the methods described herein comprise inducing T regulatory cells. In another example, nasal administration of anti-CD3 can induce IL-10 dependent Tregs that suppress inflammation and disease progression in inflammatory disease models such as autoimmune encephalomyelitis, lupus, and arthritis. In some embodiments, the methods described herein comprise inducing IL-10 dependent T regulatory cells. In some embodiments, the methods described herein comprise suppressing inflammation. In another example, administration of anti-CD3 to subjects suffering from secondary progressive multiple sclerosis, a neurodegenerative disease, is immunologically active as measured by suppression of CD8+ T cell responses and induction of CD4+IL-10 responses.

In some embodiments, the methods provided herein treat, prevent, or alleviate a symptom. In some embodiments, the symptom is at least one acute symptom. An acute symptom is a symptom that resolves in less than a month, or resolves within a timeframe that is expected for a given disease or disorder. For example, a subject suffering from a coronavirus infection (e.g. COVID-19), or a variant thereof, may experience acute symptoms such as fever, sore throat, cough, shortness of breath, and chest pain that resolve within 2 to 4 weeks.

In some embodiments, the symptom is at least one chronic symptom. A chronic symptom is a symptom that persists in a subject suffering from a disease or disorder after the time considered normal for that disease or disorder. For example, a subject suffering from a coronavirus infection (e.g. COVID-19), or a variant thereof, may experience chronic symptoms that may persist beyond 1 month, beyond 2 months, beyond 3 months, beyond 4 months, beyond 5 months, or beyond 6 months following infection. A prolonged inflammatory response can contribute to chronic symptoms. In some embodiments, chronic symptoms are at least one of fatigue, muscle aches and pains, poor sleep, cough, breathlessness, orthopnea, leg swelling, exercise intolerance due to COVID-19 induced heart failure, pulmonary embolism, pulmonary fibrosis, COVID-19-related ARDS, palpitations with mild exertion, night sweats, organ damage (e.g. cardiac or respiratory organ damage), and poor temperature control.

In some embodiments, the symptom is hyperactive immune response. A hyperactive immune response can include, for example, a cytokine release syndrome, a “cytokine storm”, or the like. In some embodiments, the hyperactive immune response comprises elevated levels of at least one of interleukin 6 (IL-6), C-reactive protein (CRP), D-dimer, interferon (IFN), interferon alpha (IFN-α), interferon gamma (IFN-γ), interleukin 1 beta (IL-1β), and/or CXCL10. In some embodiments, the hyperactive immune response comprises elevated levels of at least IL-6 and CRP. In some embodiments, the hyperactive immune response comprises elevated levels of at least IL-6. In some embodiments, the hyperactive immune response comprises elevated levels of at least CRP.

In some embodiments, the methods of treatment, preventing, or alleviating a symptom provided herein comprise measuring a symptom. In some embodiments, the symptom is hyperactive immune response. In some embodiments, measuring the hyperactive immune response comprises determining the level of at least one of interleukin 6 (IL-6), C-reactive protein (CRP), D-dimer, interferon (IFN), interferon alpha (IFN-α), interferon gamma (IFN-γ), interleukin 1 beta (IL-1β), or CXCL10. In some embodiments, measuring the hyperactive immune response comprises determining the level of at least one of IL-6, CRP, and D-dimer. In some embodiments, measuring the hyperactive immune response comprises determining the level of at least one of IL-6 and CRP. In some embodiments, measuring the hyperactive immune response comprises determining the level of at least IL-6. In some embodiments, measuring the hyperactive immune response comprises determining the level of at least CRP.

In some embodiments, the methods of treatment, preventing, or alleviating a symptom comprise administering an anti-CD3 antibody to a subject based on the hyperactive immune response in the subject. In some embodiments, the anti-CD3 antibody is administered to the subject if the hyperactive immune response comprises a high level of at least one of interleukin 6 (IL-6), C-reactive protein (CRP), D-dimer, interferon (IFN), interferon alpha (IFN-α), interferon gamma (IFN-γ), interleukin 1 beta (IL-1β), or CXCL10. In some embodiments, the anti-CD3 antibody is administered to the subject if the hyperactive immune response comprises a high level of at least one of interleukin 6 (IL-6), C-reactive protein (CRP), or D-dimer. In some embodiments, the anti-CD3 antibody is administered to the subject if the hyperactive immune response comprises a high level of at least one of interleukin 6 (IL-6) and C-reactive protein (CRP). In some embodiments, the anti-CD3 antibody is administered to the subject if the hyperactive immune response comprises a high level of at least interleukin 6 (IL-6). In some embodiments, the anti-CD3 antibody is administered to the subject if the hyperactive immune response comprises a high level of at least C-reactive protein (CRP).

A therapeutically effective amount of an active ingredient, e.g. anti-CD3 antibody of the invention, relates generally to the amount needed to achieve a therapeutic objective. As noted above, this may be a binding interaction between the antibody and its target antigen that, in certain cases, interferes with the functioning of the target. The amount required to be administered will furthermore depend on the binding affinity of the antibody for its specific antigen, and will also depend on the rate at which an administered antibody is depleted from the free volume other subject to which it is administered. Common ranges for therapeutically effective dosing of an antibody or antibody fragment of the invention may be, by way of nonlimiting example, from about 0.1 mg/kg body weight to about 50 mg/kg body weight. Common dosing frequencies may range, for example, from once daily to once a week.

The methods provided herein comprise administering an anti-CD3 as a dose or unit dose. The dose should be sufficient to result in reducing symptoms associated with a disease or disorder described herein (i.e. an effective amount). The dose chosen should be sufficient to constitute effective treatment but not as high as to cause unacceptable side effects (e.g., mucositis or anaphylactic shock). The state of the disease or disorder and the health of the patient should preferably be closely monitored during and for a reasonable period after treatment.

In some embodiments, the anti-CD3 is administered as a daily dose. In some embodiments, the anti-CD3 is administered nasally. In some embodiments, the daily dose administered nasally is 50 μg to 100 μg. In some embodiments, the daily dose is about 20 μg, about 25 μg, about 30 μg, about 35 μg, about 40 μg, about 45 μg, about 50 μg, about 55 μg, about 60 μg, about 65 μg, about 70 μg, about 75 μg, about 80 μg, about 85 μg, about 90 μg, about 95 μg, or about 100 μg. In some embodiments, the daily dose is about 50 μg.

In some embodiments, the anti-CD3 is administered as a daily dose. In some embodiments, the anti-CD3 is administered orally. In some embodiments, the daily dose administered orally is about 1.0 mg to about 2.5 mg. In some embodiments, the daily dose administered orally is about 0.5 mg, about 0.75 mg, about 1.0 mg, about 1.25 mg, about 1.5 mg, about 1.75 mg, about 2.0 mg, about 2.25 mg, about 2.5 mg, about 2.75 mg, about 3.0 mg, about 3.25 mg, about 3.5 mg, about 3.75 mg, about 4.0 mg, about 4.25 mg, about 4.5 mg, about 4.75 mg, or about 5.0 mg.

In some embodiments, the anti-CD3 is administered for at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24, days, at least 25 days, at least 26 days, at least 27 days, at least 28 days, at least 29 days, or at least 30 days. In some embodiments, the anti-CD3 is administered for at least 10 consecutive days.

The anti-CD3 can be administered by nasal drops, nasal inhalation, inhalation through the mouth, intravenously, orally, any combination thereof or any other route of administration described herein. Alternatively the, active compound is administered orally via an enteric-coated capsule. Administration by inhalation may be in the form of an inhaler or a nebulizer. The nebulizer and/or inhaler is handheld. Optionally, the nebulizer and/or inhaler can be of different sizes to fit children and/or adults.

Efficaciousness of treatment is determined in association with any known method for diagnosing or treating a disease or disorder described herein. Alleviation of one or more symptoms with the disease or disorder indicates that the antibody confers a clinical benefit.

### Co-Administration of Anti-CD3 and Other Agents

The disclosure provides methods of treating, preventing, or alleviating a symptom of a disease or disorder in a subject in need thereof comprising co-administering to the subject a composition comprising an CD3 antibody (anti-CD3) and another active agent. An active agent can be any pharmaceutical or supplement for treating a disease or disorder. The disease or disorder can be any disease or disorder provided herein. In some embodiments, the disease or disorder is a coronavirus infection, inflammatory disease, acute respiratory distress syndrome (ARDS), neurodegenerative, or a pulmonary disease in a subject. Co-administration of the anti-CD3 and another active agent can be in any order. For example, the anti-CD3 can be administered before, simultaneously, or after another active agent. Co-administration occurs at any time during the treatment period at which the subject in need thereof receives treatment for the disease. Co-administration of anti-CD3 and another active agent may be on the same day, separate days, the same week, or separate weeks.

In some embodiments, the methods of treatment, preventing, or alleviating a symptom provided herein comprise co-administering anti-CD3 antibodies with another therapeutic agent. In some embodiments, the anti-CD3 antibodies are co-administered with dexamethasone. In some embodiments, the anti-CD3 antibodies of the disclosure are administered before, simultaneously, or following administration of dexamethasone. When administered simultaneously the anti-CD3 antibodies and the dexamethasone can be formulated together or separately and administered as described herein.

In some embodiments, the methods of treatment, preventing, or alleviating a symptom provided herein comprise co-administering the anti-CD3 antibodies with a monoclonal antibody. In some embodiments, the monoclonal antibody is an anti-IL-6R antibody. In some embodiments, the antibody is tocilizumab. In some embodiments, the IL-6R antibody comprises a VH CDR1 region comprising the amino acid sequence of SEQ ID NO: 15, a VH CDR2 region comprising the amino acid sequence of SEQ ID NO: 37, a VH CDR3 region comprising the amino acid sequence of SEQ ID NO: 35, a VL CDR1 region comprising the amino acid sequence of SEQ ID NO: 24, a VL CDR2 region comprising the amino acid sequence of SEQ ID NO: 25, and a VL CDR3 region comprising the amino acid sequence of SEQ ID NO: 26. In some embodiments, the methods of treatment, preventing, or alleviating a symptom provided herein include co-administering anti-CD3 delivered intranasally and anti-IL6R antibodies. In some embodiments, the anti-IL6R antibodies are delivered by intravenously. In some embodiments, the anti-IL6R antibodies are delivered by inhalation. In some embodiments, the anti-IL6R antibodies are delivered orally. In some embodiments, the anti-IL6R antibodies are delivered subcutaneously. In some embodiments, administration of an anti-IL-6R mAb and anti-CD3 produces a synergistic effect that reduces symptoms associated with a disease or disorder described herein, such as a coronavirus disease (e.g. COVID-19, SARS, or MERS).

In some embodiments, the methods of treatment, preventing, or alleviating a symptom provided herein comprise co-administering the anti-CD3 antibody and an anti-TNFα antibody, an anti-CD20 antibody an anti-IFNγ antibody, an anti-Granulocyte-Macrophage Colony-Stimulating Factor antibody, an anti-IL6R antibody, or combinations thereof. In some embodiments, the anti-CD3 is co-administered with casirivimab and imdevimab. In some embodiments, the anti-CD3 is co-administered with bamlanivimab and etesevimab. In some embodiments, the anti-CD3 is co-administered with sotrovimab.

In some embodiments, the methods of treatment, preventing, or alleviating a symptom provided herein comprise co-administering the anti-CD3 antibody and an antiviral. In some embodiments, the antiviral is azidothymidine (also called zidovudine or AZT), remdesivir, or actinomycin D. In some embodiments, the methods of treatment, preventing, or alleviating a symptom provided herein comprise co-administering the anti-CD3 antibody and an immune booster drug, vitamin C, vitamin D, vitamin E, or any combination thereof. In some embodiments, the methods of treatment, preventing, or alleviating a symptom provided herein comprise co-administering the anti-CD3 antibody an anticoagulation agent (i.e. blood thinner). In some embodiments, the anticoagulation agent is low dose heparin or enoxaparin.

In some embodiments, the methods of treatment, preventing, or alleviating a symptom provided herein comprise co-administering the anti-CD3 antibody and a corticosteroid. In some embodiments, the corticosteroid is dexamethasone, prednisone, or methylprednisolone. In some embodiments, the corticosteroid is dexamethasone. In some embodiments, dexamethasone is administered orally as a unit dose. In some embodiments, dexamethasone is administered once per week, twice per week, three times per week, or four times per week for the duration of treatment. In some embodiments, dexamethasone is administered orally as a unit dose, wherein the unit dose is about 1 mg, about 2 mg, about 3 mg, about 4 mg, about 5 mg, about 6 mg, about 7 mg, about 8 mg, about 9 mg, or about 10 mg. In some embodiments, dexamethasone is administered orally as a unit dose, wherein the unit dose is about 6 mg.

In some embodiments, the methods of treatment, preventing, or alleviating a symptom provided herein comprise co-administering the anti-CD3 antibody and convalescent plasma. In some embodiments, the convalescent plasma is derived from a subject that has recovered from a coronavirus.

### Coronavirus Infection

The disclosure provides methods of treating, preventing, or alleviating a symptom of a coronavirus infection in a subject in need thereof comprising administering to the subject a composition comprising an anti-CD3 antibody.

In some embodiments, the subject has a disease or pathology associated with coronavirus infection. For example, the coronavirus can be SARS-CoV (i.e. SARS), SARS-CoV-2 (i.e. COVID-19), MERS-CoV (i.e. MERS), or a mutant and/or variant thereof. In some embodiments, the subject has a disease of pathology associated with MERS and/or its variants. In some embodiments, the subject has a disease or pathology associated with SARS and/or its variants. In some embodiments, the subject has a disease or pathology associated with SARS-CoV-2 and/or its variants. “Variants” refers to genetic variants of a coronavirus, such that novel genetic mutations have occurred in the variant in relation to one or more known strains of the coronavirus. Mutations (e.g. substitutions or deletions) can be to any nucleotide in the genome of the coronavirus. The variants can be variants of interest, variants of concern, or variants of high consequence. For example the B.1.1.7 (Alpha), B.1.351 (Beta), B.1.617 (Delta), and P.1 (Gamma), B.1.526 (Iota), B.1.427 (Epsilon), B.1.429 (Epsilon), B.1.1.7 (Alpha), P.2 (Zeta), and their lineages are currently classified as variants of SARS-CoV-2. It is to be understood that new variants of coronavirus with novel mutations or sets of mutations can arise, and these are also covered by the term “coronavirus” as referred to herein.

The methods provided herein comprise administering an anti-CD3 as a dose or unit dose. The dose should be sufficient to result in reducing symptoms associated with a coronavirus infection (e.g. hyperactive immune response or cytokine storm) or slowing the replication of the coronavirus within the host and also preferably prevent or reduce the symptoms of a coronavirus-related disease (e.g. COVID-19, SARS, or MERS). The dose chosen should be sufficient to constitute effective treatment but not as high as to cause unacceptable side effects (e.g., mucositis or anaphylactic shock). The state of the disease condition (e.g., SERS, MERS, or COVID-19) and the health of the patient should preferably be closely monitored during and for a reasonable period after treatment.

In some embodiments, the anti-CD3 is administered as a daily dose. In some embodiments, the anti-CD3 is administered nasally. In some embodiments, the daily dose administered nasally is 50 μg to 100 μg. In some embodiments, the daily dose is about 20 μg, about 25 μg, about 30 μg, about 35 μg, about 40 μg, about 45 μg, about 50 μg, about 55 μg, about 60 μg, about 65 μg, about 70 μg, about 75 μg, about 80 μg, about 85 μg, about 90 μg, about 95 μg, or about 100 μg. In some embodiments, the daily dose is about 50 μg.

In some embodiments, the anti-CD3 is administered as a daily dose. In some embodiments, the anti-CD3 is administered orally. In some embodiments, the daily dose administered orally is about 1.0 mg to about 2.5 mg. In some embodiments, the daily dose administered orally is about 0.5 mg, about 0.75 mg, about 1.0 mg, about 1.25 mg, about 1.5 mg, about 1.75 mg, about 2.0 mg, about 2.25 mg, about 2.5 mg, about 2.75 mg, about 3.0 mg, about 3.25 mg, about 3.5 mg, about 3.75 mg, about 4.0 mg, about 4.25 mg, about 4.5 mg, about 4.75 mg, or about 5.0 mg.

In some embodiments, the anti-CD3 is administered for at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24, days, at least 25 days, at least 26 days, at least 27 days, at least 28 days, at least 29 days, or at least 30 days. In some embodiments, the anti-CD3 is administered for at least 10 consecutive days.

In some embodiments, the anti-CD3 antibody is co-administered with a corticosteroid. In some embodiments, the corticosteroid is dexamethasone, prednisone, or methylprednisolone. In some embodiments, the corticosteroid is dexamethasone. In some embodiments, dexamethasone is administered orally as a unit dose. In some embodiments, dexamethasone is administered once per week, twice per week, three times per week, or four times per week for the duration of treatment. In some embodiments, dexamethasone is administered orally. In some embodiments, dexamethasone is administered nasally. In some embodiments, dexamethasone is administered as a unit dose, wherein the unit dose is about 1 mg, about 2 mg, about 3 mg, about 4 mg, about 5 mg, about 6 mg, about 7 mg, about 8 mg, about 9 mg, or about 10 mg. In some embodiments, dexamethasone is administered orally as a unit dose, wherein the unit dose is about 6 mg.

In some embodiments, the anti-CD3 antibody is co-administered with an antiviral. In some embodiments, the antiviral is zidovudine. In some embodiments, the zivudine is administered orally as a unit dose. In some embodiments, the unit dose is about 100 mg, about 150 mg, about 200 mg, about 250 mg, about 300 mg, about 350 mg, about 400 mg, about 450 mg, about 500 mg, about 550 mg, or about 600 mg.

In some embodiments, the subject is a human subject. In some embodiments, the subject has or is suspected of having a coronavirus infection. In some embodiments, the subject has been or thought to have been exposed to a coronavirus and has not yet developed symptoms of a coronavirus infection. In some embodiments, the coronavirus is, for example, the virus that causes COVID-19, SARS, or MERS. In some embodiments, a sample from a subject is tested for active coronavirus, evidence of coronavirus exposure, or evidence of coronavirus immunity. Samples from the subject can be, for example, a blood sample, a serum or plasma sample, a lavage sample, a urine sample, or any sample useful for determining a hyperactive immune response. In some embodiments, the subject has tested positive in a coronavirus diagnostic test. In some embodiments, the subject has an active coronavirus infection. In some embodiments, the diagnostic test measures presence of the coronavirus genome. In some embodiments, the diagnostic test is a quantitative polymerase chain reaction test. In some embodiments, the diagnostic test measures the presence of antibodies that bind to a coronavirus epitope. In some embodiments, the coronavirus epitope is on the coronavirus spike protein.

Signs and symptoms of a coronavirus infection can be determined by a medical professional or self-reported in a symptomology survey as a patient reported outcomes survey. In some embodiments, the symptom is one or more of fever, gastrointestinal symptoms, respiratory symptoms, anosmia (loss of smell), dysgeusia (loss of taste), cough, headache, throat ache, pain when swallowing, dyspnea, difficult breathing, shortness of breath, nausea, vomiting, reduced 02 saturation, diarrhea, rhinorrhea, abdominal pain, myalgia, fever, conjunctivitis, and loss of appetite.

Coronavirus infection may cause a hyperactive immune response as a symptom, sometimes referred to as cytokine storm, hyperinflammatory response, or the like. The hyperactive immune response is characterized by a rapid increase and elevated (i.e. high) levels of pro-inflammatory molecules such as cytokines and chemokines. The rapid increase and high levels of pro-inflammatory molecules can include interleukin 6 (IL-6), C-reactive protein, D-dimer, interferon (IFN), interferon alpha (IFN-α), interferon gamma (IFN-γ), interleukin 1 beta (IL-1β), and/or CXCL10. Without being bound by theory, it is thought that the hyperactive immune response contributes to serious and fatal cases of coronavirus infection.

In some embodiments, the symptom is hyperactive immune response. In some embodiments, the hyperactive immune response comprises elevated levels of at least one of interleukin 6 (IL-6), C-reactive protein (CRP), D-dimer, interferon (IFN), interferon alpha (IFN-α), interferon gamma (IFN-γ), interleukin 1 beta (IL-1β), and/or CXCL10. In some embodiments, the hyperactive immune response comprises elevated levels of at least IL-6 and CRP. In some embodiments, the hyperactive immune response comprises elevated levels of at least IL-6. In some embodiments, the hyperactive immune response comprises elevated levels of at least CRP.

Hyperactive immune response can be measured by determining levels of pro-inflammatory molecules such as cytokines and chemokines in a sample collected from a subject. Samples from the subject can be, for example, a blood sample, a serum or plasma sample, a lavage sample, a urine sample, or any sample useful for determining a hyperactive immune response. Any method known in the art to measure cytokines and chemokines in a sample collected from a subject may be used. For example, the method can be, without limitation, cytometric bead arrays, quantitative polymerase chain reaction, immunoassays (e.g. ELISA or immunoturbidimetry assays), electrochemiluminescence immunoassays, or the like. Commercially available laboratory developed tests and diagnostic kits for determining the level of cytokines or chemokines may be used. General guidelines available to those skilled in the art can be used to determine if a level of a cytokine or chemokine (e.g. IL-6 or CRP) is low, normal, or elevated (i.e. high). It is understood that data provided by the methods used to determine the level of one or more cytokines or chemokines is interpreted by the skilled artisan (e.g. a practicing physician) in the context of the condition of a particular patient.

In some embodiments, the methods of treatment, preventing, or alleviating a symptom provided herein comprise measuring a symptom. In some embodiments, the symptom is hyperactive immune response. In some embodiments, measuring the hyperactive immune response comprises determining the level of at least one of interleukin 6 (IL-6), C-reactive protein (CRP), D-dimer, interferon (IFN), interferon alpha (IFN-α), interferon gamma (IFN-γ), interleukin 1 beta (IL-1β), or CXCL10. In some embodiments, measuring the hyperactive immune response comprises determining the level of at least one of IL-6, CRP, and D-dimer. In some embodiments, measuring the hyperactive immune response comprises determining the level of at least one of IL-6 and CRP. In some embodiments, measuring the hyperactive immune response comprises determining the level of at least IL-6. In some embodiments, measuring the hyperactive immune response comprises determining the level of at least CRP.

In some embodiments, the methods of treatment, preventing, or alleviating a symptom comprise administering an anti-CD3 antibody to a subject based on the hyperactive immune response in the subject. In some embodiments, the anti-CD3 antibody is administered to the subject if the hyperactive immune response comprises a high level of at least one of interleukin 6 (IL-6), C-reactive protein (CRP), D-dimer, interferon (IFN), interferon alpha (IFN-α), interferon gamma (IFN-γ), interleukin 1 beta (IL-1β), or CXCL10. In some embodiments, the anti-CD3 antibody is administered to the subject if the hyperactive immune response comprises a high level of at least one of interleukin 6 (IL-6), C-reactive protein (CRP), or D-dimer. In some embodiments, the anti-CD3 antibody is administered to the subject if the hyperactive immune response comprises a high level of at least one of interleukin 6 (IL-6) and C-reactive protein (CRP). In some embodiments, the anti-CD3 antibody is administered to the subject if the hyperactive immune response comprises a high level of at least interleukin 6 (IL-6). In some embodiments, the anti-CD3 antibody is administered to the subject if the hyperactive immune response comprises a high level of at least C-reactive protein (CRP).

### Other Diseases and Disorders

Provided herein are methods of treatment, preventing, or alleviating a symptom of a disease or disorder in a subject in need thereof comprising administering to the subject a composition comprising an CD3 antibody (anti-CD3). In some embodiments, the subject has an inflammatory disease or disorder. In some embodiments, the inflammatory disease is autoimmune encephalomyelitis. In some embodiments, the inflammatory disease is lupus. In some embodiments, the inflammatory disease is arthritis. In some embodiments, the subject has acute respiratory distress syndrome (ARDS). In some embodiments, the subject has a disease or pathology associated with a neurodegenerative disease or disorder. In some embodiments, the neurodegenerative disorder is multiple sclerosis. In some embodiments, the neurodegenerative disorder is secondary progressive multiple sclerosis. In some embodiments, the subject has a disease or pathology associated with a pulmonary disease or disorder. Examples of pulmonary inflammatory diseases include ARDS (acute respiratory distress syndrome) and systemic pulmonary sclerosis.

### Pharmaceutical Compositions

The anti-CD3 antibodies described herein can be incorporated into a pharmaceutical composition suitable for oral or mucosal administration, e.g., by ingestion, inhalation, or absorption, e.g., via nasal, intranasal, pulmonary, buccal, sublingual, rectal, or vaginal administration. Such compositions can include an inert diluent or an edible carrier. For the purpose of oral therapeutic administration, the active compound (e.g., an anti-CD3 antibody) can be incorporated with excipients and used in solid or liquid (including gel) form. Oral anti-CD3 antibody compositions can also be prepared using an excipient. Pharmaceutically compatible binding agents, and/or adjuvant materials can be included as part of the composition. Oral dosage forms comprising anti-CD3 antibody are provided, wherein the dosage forms, upon oral administration, provide a therapeutically effective blood level of anti-CD3 antibody to a subject. Also provided are mucosal dosage forms comprising anti-CD3 antibody wherein the dosage forms, upon mucosal administration, provide a therapeutically effective blood level of anti-CD3 antibody to a subject. For the purpose of mucosal therapeutic administration, the active compound (e.g., an anti-CD3 antibody) can be incorporated with excipients or carriers suitable for administration by inhalation or absorption, e.g., via nasal sprays or drops, or rectal or vaginal suppositories.

Solid oral dosage forms include, but are not limited to, tablets (e.g., chewable tablets), capsules, caplets, powders, pellets, granules, powder in a sachet, enteric coated tablets, enteric coated beads, and enteric coated soft gel capsules. Also included are multi-layered tablets, wherein different layers can contain different drugs. Solid dosage forms also include powders, pellets and granules that are encapsulated. The powders, pellets, and granules can be coated, e.g., with a suitable polymer or a conventional coating material to achieve, for example, greater stability in the gastrointestinal tract, or to achieve a desired rate of release. In addition, a capsule comprising the powder, pellets or granules can be further coated. A tablet or caplet can be scored to facilitate division for ease in adjusting dosage as needed. The dosage forms of the present invention can be unit dosage forms wherein the dosage form is intended to deliver one therapeutic dose per administration, e.g., one tablet is equal to one dose. Such dosage forms can be prepared by methods of pharmacy well known to those skilled in the art (see Remington's Pharmaceutical Sciences, 18th ed., Mack Publishing, Easton Pa. (1990)).

Typical oral dosage forms can be prepared by combining the active ingredients in an intimate admixture with at least one excipient according to conventional pharmaceutical compounding techniques. Excipients can take a wide variety of forms depending on the form of preparation desired for administration. For example, excipients suitable for use in solid oral dosage forms (e.g., powders, tablets, capsules, and caplets) include, but are not limited to, starches, sugars, micro-crystalline cellulose, diluents, granulating agents, lubricants, binders, and disintegrating agents. Examples of excipients suitable for use in oral liquid dosage forms include, but are not limited to, water, glycols, oils, alcohols, flavoring agents, preservatives, and coloring agents.

Tablets and capsules represent convenient pharmaceutical compositions and oral dosage forms, in which case solid excipients are employed. If desired, tablets can be coated by standard aqueous or non-aqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers, or both, and then shaping the product into the desired presentation if necessary.

As one example, a tablet can be prepared by compression or by molding. Compressed tablets can be prepared, e.g., by compressing, in a suitable machine, the active ingredients (e.g., an anti-CD3 antibody) in a free-flowing form such as powder or granules, optionally mixed with an excipient. Molded tablets can be made, e.g., by molding, in a suitable machine, a mixture of the powdered anti-CD3 antibody compound moistened, e.g., with an inert liquid diluent.

Excipients that can be used in oral dosage forms of the invention include, but are not limited to, binders, fillers, disintegrants, and lubricants. Binders suitable for use in pharmaceutical compositions and dosage forms include, but are not limited to, corn starch, potato starch, or other starches, gum tragacanth or gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidinones, methyl cellulose, pre-gelatinized starch, hydroxypropyl methyl cellulose, (e.g., Nos. 2208, 2906, 2910), microcrystalline cellulose, and mixtures thereof.

Suitable forms of microcrystalline cellulose include, but are not limited to, the materials sold as AVICEL™ PH-101, AVICEL™ PH-103, AVICEL™. RC-581, AVICEL™PH-105 (available from FMC Corporation, American Viscose Division, Avicel Sales, Marcus Hook, Pa.), and mixtures thereof. A specific binder is a mixture of microcrystalline cellulose and sodium carboxymethyl cellulose sold as AVICEL™ RC-581. Suitable anhydrous or low moisture excipients or additives include AVICEL™ PH-103 and Starch 1500 ™LM.

Examples of fillers suitable for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. The binder or filler in pharmaceutical compositions and dosage forms of the invention is typically present in from about 50 to about 99 weight percent of the pharmaceutical composition or dosage form.

Disintegrants can be used in the pharmaceutical compositions and oral or mucosal dosage forms of the invention to provide tablets that disintegrate when exposed to an aqueous environment. Tablets containing too much disintegrant might disintegrate in storage, while those containing too little might not disintegrate at a desired rate or under desired conditions. Thus, a sufficient amount of disintegrant that is neither too much nor too little to detrimentally alter the release of the active ingredients should be used to form the pharmaceutical compositions and solid oral dosage forms described herein. The amount of disintegrant used varies based upon the type of formulation, and is readily discernible to those of ordinary skill in the art. Typically, pharmaceutical compositions and dosage forms comprise from about 0.5 to about 15 weight percent of disintegrant, preferably from about 1 to about 5 weight percent of disintegrant.

Disintegrants that can be used in pharmaceutical compositions and oral or mucosal dosage forms of the invention include, but are not limited to, agar-agar, alginic acid, calcium carbonate, Primogel, microcrystalline cellulose, croscarmellose sodium, crospovidone, polacrilin potassium, sodium starch glycolate, corn, potato or tapioca starch, other starches, pre-gelatinized starch, other starches, clays, other algins, other celluloses, gums, and mixtures thereof.

Lubricants that can be used in pharmaceutical compositions and dosage forms of the invention include, but are not limited to, calcium stearate, magnesium stearate or Sterotes, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSIL™200, manufactured by W. R. Grace Co. of Baltimore, Md.), a coagulated aerosol of synthetic silica (marketed by Degussa Co. of Plano, Tex.), CAB-O-SIL™(a pyrogenic silicon dioxide product sold by Cabot Co. of Boston, Mass.), and mixtures thereof. If used at all, lubricants are typically used in an amount of less than about 1 weight percent of the pharmaceutical compositions or dosage forms into which they are incorporated. A glidant such as colloidal silicon dioxide can also be used.

The pharmaceutical compositions and oral or mucosal dosage forms can further comprise one or more compounds that reduce the rate by which an active ingredient will decompose. Thus the oral dosage forms described herein can be processed into an immediate release or a sustained release dosage form. Immediate release dosage forms may release the anti-CD3 antibody in a fairly short time, for example, within a few minutes to within a few hours. Sustained release dosage forms may release the anti-CD3 antibody over a period of several hours, for example, up to 24 hours or longer, if desired. In either case, the delivery can be controlled to be substantially at a certain predetermined rate over the period of delivery. In some embodiments, the solid oral dosage forms can be coated with a polymeric or other known coating material(s) to achieve, for example, greater stability on the shelf or in the gastrointestinal tract, or to achieve control over drug release. Such coating techniques and materials used therein are known in the art. Such compounds, which are referred to herein as “stabilizers,” include, but are not limited to, antioxidants such as ascorbic acid and salt buffers. For example, cellulose acetate phthalate, polyvinyl acetate phthalate, hydroxypropylmethyl cellulose phthalate, methacrylic acid-methacrylic acid ester copolymers, cellulose acetate trimellitate, carboxymethylethyl cellulose, and hydroxypropylmethyl cellulose acetate succinate, among others, can be used to achieve enteric coating. Mixtures of waxes, shellac, zein, ethyl cellulose, acrylic resins, cellulose acetate, silicone elastomers can be used to achieve sustained release coating. See, for example, Remington, supra, Chapter 93, for other types of coatings, techniques and equipment.

Liquids for oral or mucosal administration represent another convenient dosage form, in which case a solvent can be employed. In some embodiments, the solvent is a buffered liquid such as phosphate buffered saline (PBS). Liquid oral dosage forms can be prepared by combining the active ingredient in a suitable solvent to form a solution, suspension, syrup, or elixir of the active ingredient in the liquid. The solutions, suspensions, syrups, and elixirs may optionally comprise other additives including, but not limited to, glycerin, sorbitol, propylene glycol, sugars or other sweeteners, flavoring agents, and stabilizers. Flavoring agents can include, but are not limited to peppermint, methyl salicylate, or orange flavoring. Sweeteners can include sugars, aspartame, saccharin, sodium cyclamate and xylitol.

In order to reduce the degree of inactivation of orally administered anti-CD3 antibody in the stomach of the treated subject, an antiacid can be administered simultaneously with the immunoglobulin, which neutralizes the otherwise acidic character of the gut. Thus in some embodiments, the anti-CD3 antibody is administered orally with an antacid, e.g., aluminum hydroxide or magnesium hydroxide such as MAALOX™antacid or MYLANTA™. antacid, or an H2 blocker, such as cimetidine or ranitidine. One of skill in the art will appreciate that the dose of antacid administered in conjunction with an anti-CD3 antibody depends on the particular antacid used. When the antacid is MYLANTA™ antacid in liquid form, between 15 ml and 30 ml can be administered, e.g., about 15 ml. When the cimetidine H2 blocker is used, between about 400 and 800 mg per day can be used.

The kits described herein can include an anti-CD3 antibody composition as an already prepared liquid oral or mucosal dosage form ready for administration or, alternatively, can include an anti-CD3 antibody composition as a solid pharmaceutical composition that can be reconstituted with a solvent to provide a liquid oral dosage form or mucosal dosage form. When the kit includes an anti-CD3 antibody composition as a solid pharmaceutical composition that can be reconstituted with a solvent to provide a liquid dosage form (e.g., for oral or nasal administration), the kit may optionally include a reconstituting solvent. In this case, the constituting or reconstituting solvent is combined with the active ingredient to provide a liquid oral dosage form of the active ingredient. Typically, the active ingredient is soluble in the solvent and forms a solution. The solvent can be, e.g., water, a non-aqueous liquid, or a combination of a non-aqueous component and an aqueous component. Suitable non-aqueous components include, but are not limited to oils; alcohols, such as ethanol; glycerin; and glycols, such as polyethylene glycol and propylene glycol. In some embodiments, the solvent is phosphate buffered saline (PBS).

For administration by inhalation, the mucosal anti-CD3 antibody compounds can be delivered in the form of an aerosol spray from pressured container or dispenser that contains a suitable propellant, e.g., a gas such as carbon dioxide, or a nebulizer. Such methods include those described in U.S. Pat. No. 6,468,798.

Systemic administration can also be by transmucosal means. For transmucosal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal drops or sprays, or rectal or vaginal suppositories.

The anti-CD3 antibody compounds can also be prepared in the form of suppositories (e.g., with conventional suppository bases such as cocoa butter and other glycerides) or retention enemas for rectal delivery.

In one embodiment, the oral or mucosal anti-CD3 antibody compositions are prepared with carriers that will protect the anti-CD3 antibody against rapid elimination from the body, such as a controlled release formulation, including implants and microencapsulated delivery systems.

Biodegradable, biocompatible polymers can be used, such as ethylene vinyl acetate, polyanhydrides, polyglycolic acid, collagen, polyorthoesters, and polylactic acid. Such formulations can be prepared using standard techniques. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

Dosage, toxicity and therapeutic efficacy of such anti-CD3 antibody compositions can be determined by standard pharmaceutical procedures in cell cultures (e.g., of cells taken from an animal after mucosal administration of an anti-CD3 antibody) or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compositions which exhibit high therapeutic indices are preferred. While anti-CD3 antibody compositions that exhibit toxic side effects may be used, care should be taken to design a delivery system that targets such compounds to the site of affected tissue in order to minimize potential damage and, thereby, reduce side effects.

The data obtained from the cell cultures (e.g., of cells taken from an animal after mucosal administration of an anti-CD3 antibody) and animal studies can be used in formulating a range of dosage for use in humans. The dosage of anti-CD3 antibody compositions lies preferably within a range of circulating concentrations that include the ED50 with little or no toxicity. The dosage may vary within this range depending upon the dosage form employed and the route of administration utilized. For any oral or mucosal anti-CD3 antibody compositions used in the methods described herein, the therapeutically effective dose can be estimated initially from assays of cell cultures (e.g., of cells taken from an animal after mucosal administration of an anti-CD3 antibody). A dose may be formulated in animal models to achieve a desired circulating plasma concentration of IL-10 or TGFβ, or of regulatory cells, in the range that includes the IC50 (i.e., the concentration of the test compound which achieves a half-maximal inhibition of symptoms) as determined in cell culture. Such information can be used to more accurately determine useful doses in humans. Levels of IL-10 or TGFβ in plasma can be measured by methods known in the art, for example, by ELISA. Levels of regulatory cells can be measured by methods known in the art, for example, by flow cytometry-based methods.

As defined herein, a therapeutically effective amount of an anti-CD3 antibody (i.e., an effective dosage) depends on the antibody selected, the mode of delivery, and the condition to be treated. For instance, single dose amounts in the range of approximately 1: g/kg to 1000 g/kg may be administered; in some embodiments, about 5, 10, 50, 100, or 500:g/kg may be administered. In some embodiments, e.g., pediatric subjects, about 1 to 100: g/kg, e.g., about 25 or 50: g/kg, of anti-CD3 antibody can be administered. The anti-CD3 antibody compositions can be administered from one or more times per day to one or more times per week; including once every other day. The oral or mucosal anti-CD3 antibody compositions can be administered, e.g., for about 10 to 14 days or longer. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the compounds can include a single treatment or, can include a series of treatments.

The oral or mucosal anti-CD3 antibody compositions can also include one or more therapeutic agents useful for treating a coronaviral infection. Such therapeutic agents can include, e.g., NSAIDs (including COX-2 inhibitors); other antibodies, e.g., anti-cytokine antibodies, e.g., antibodies to IFN-α, IFN □ and/or TNFα.; gold-containing compounds; immunosuppressive drugs (such as corticosteroids, e.g., dexamethasone, prednisolone and methyl prednisolone).

For example, the combination therapy can include one or more antibodies of the invention coformulated with, and/or coadministered with, one or more additional therapeutic agents, such as corticosteroids, (e.g., dexamethasone, prednisolone and methyl prednisolone) antiviral drugs, immune booster drugs, Vitamin C, Vitamin D, Vitamin E. Such combination therapies may advantageously utilize lower dosages of the administered therapeutic agents, thus avoiding possible toxicities or complications associated with the various monotherapies.

The anti-viral drug is Azidothymidine, Remdesivir, or Actinomycin D.

In some embodiments, a subject is administered anti-CD3 antibody delivered orally, nasally or both orally and nasally.

In some embodiments, a subject is administered anti-CD3 antibody and a corticosteroid such as dexamethasone, delivered orally.

In some embodiments, a subject is administered anti-CD3 antibody nasally and a corticosteroid such as dexamethasone is delivered orally, such as for example by inhalation.

In some embodiments, a subject is administered anti-CD3 antibody nasally and orally and a corticosteroid such as dexamethasone is delivered orally such as for example by inhalation.

The pharmaceutical compositions can be included in a container, pack, or dispenser together with instructions for administration.

The methods of treatment or prevention typically include administering to a subject an oral or mucosal anti-CD-3 antibody composition sufficient to stimulate the mucosal immune system. In some embodiments, the methods include administering an oral or mucosal anti-CD3 antibody composition sufficient to increase IL-10 and/or TGF-.beta. production by T cells in the peripheral blood, e.g., regulatory T cells, e.g., by about 100%, 200%, 300% or more. In some embodiments, the methods include administering an oral anti-CD3 antibody composition sufficient to decrease T cell proliferation in the peripheral blood, e.g., by about 20%; e.g., in some embodiments, by at least about 20%, 30%, 40%, 50%, 60%, 70%, 80%, 90%, or more.

In some embodiments, the methods can include administering to the subject an dexamethazone, before, concomitantly with, or after administration of the oral or mucosal anti-CD3 compositions.

### Definitions

Unless otherwise defined, scientific and technical terms used in connection with the present disclosure shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well-known and commonly used in the art. Standard techniques are used for recombinant DNA, oligonucleotide synthesis, and tissue culture and transformation (e.g., electroporation, lipofection). Enzymatic reactions and purification techniques are performed according to manufacturer's specifications or as commonly accomplished in the art or as described herein. The foregoing techniques and procedures are generally performed according to conventional methods well known in the art and as described in various general and more specific references that are cited and discussed throughout the present specification. See e.g., Sambrook et al. Molecular Cloning: A Laboratory Manual (2d ed., Cold Spring Harbor Laboratory Press, Cold Spring Harbor, N.Y. (1989)). The nomenclatures utilized in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

As utilized in accordance with the present disclosure, the following terms, unless otherwise indicated, shall be understood to have the following meanings:

As used herein, the term “antibody” refers to immunoglobulin molecules and immunologically active portions of immunoglobulin (Ig) molecules, i.e., molecules that contain an antigen binding site that specifically binds (immunoreacts with) an antigen. Such antibodies include, but are not limited to, polyclonal, monoclonal, chimeric, single chain, Fab, Fab′ and F(ab′)2 fragments, and an Fab expression library. By “specifically bind” or “immunoreacts with” is meant that the antibody reacts with one or more antigenic determinants of the desired antigen and does not react (i.e., bind) with other polypeptides or binds at much lower affinity (Kd>10-6) with other polypeptides.

The basic antibody structural unit is known to comprise a tetramer. Each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. Human light chains are classified as kappa and lambda light chains. Heavy chains are classified as mu, delta, gamma, alpha, or epsilon, and define the antibody's isotype as IgM, IgD, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ea., 2nd ed. Raven Press, N.Y. (1989)). The variable regions of each light/heavy chain pair form the antibody binding site.

The term “monoclonal antibody” (MAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs contain an antigen binding site capable of immunoreacting with a particular epitope of the antigen characterized by a unique binding affinity for it.

In general, antibody molecules obtained from humans relate to any of the classes IgG, IgM, IgA, IgE and IgD, which differ from one another by the nature of the heavy chain present in the molecule. Certain classes have subclasses as well, such as IgG1, IgG2, and others. Furthermore, in humans, the light chain may be a kappa chain or a lambda chain.

As used herein, the term “epitope” includes any protein determinant capable of specific binding to an immunoglobulin, a scFv, or a T-cell receptor. The term “epitope” includes any protein determinant capable of specific binding to an immunoglobulin or T-cell receptor. Epitopic determinants usually consist of chemically active surface groupings of molecules such as amino acids or sugar side chains and usually have specific three dimensional structural characteristics, as well as specific charge characteristics. An antibody is said to specifically bind an antigen when the dissociation constant is ≤1 μM; preferably ≤100 nM and most preferably ≤10 nM.

As used herein, the terms “immunological binding” and “immunological binding properties” and “specific binding” refer to the non-covalent interactions of the type which occur between an immunoglobulin molecule and an antigen for which the immunoglobulin is specific. The strength, or affinity of immunological binding interactions can be expressed in terms of the dissociation constant (Kd) of the interaction, wherein a smaller Kd represents a greater affinity. Immunological binding properties of selected polypeptides are quantified using methods well known in the art. One such method entails measuring the rates of antigen-binding site/antigen complex formation and dissociation, wherein those rates depend on the concentrations of the complex partners, the affinity of the interaction, and geometric parameters that equally influence the rate in both directions. Thus, both the “on rate constant” (Kon) and the “off rate constant” (Koff) can be determined by calculation of the concentrations and the actual rates of association and dissociation. (See Nature 361:186-87 (1993)). The ratio of Koff/Kon enables the cancellation of all parameters not related to affinity, and is equal to the dissociation constant Kd. (See, generally, Davies et al. (1990) Annual Rev Biochem 59:439-473). An antibody of the present disclosure is said to specifically bind to a CD3 epitope when the equilibrium binding constant (Kd) is about 1 μM, preferably about 100 nM, more preferably about 10 nM, and most preferably about 100 pM to about 1 pM, as measured by assays such as radioligand binding assays or similar assays known to those skilled in the art.

Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine valine, glutamic-aspartic, and asparagine-glutamine.

As discussed herein, minor variations in the amino acid sequences of antibodies or immunoglobulin molecules are contemplated as being encompassed by the present disclosure, providing that the variations in the amino acid sequence maintain at least 75%, more preferably at least 80%, 90%, 95%, and most preferably 99%. In particular, conservative amino acid replacements are contemplated. Conservative replacements are those that take place within a family of amino acids that are related in their side chains. Genetically encoded amino acids are generally divided into families: (1) acidic amino acids are aspartate, glutamate; (2) basic amino acids are lysine, arginine, histidine; (3) non-polar amino acids are alanine, valine, leucine, isoleucine, proline, phenylalanine, methionine, tryptophan, and (4) uncharged polar amino acids are glycine, asparagine, glutamine, cysteine, serine, threonine, tyrosine. The hydrophilic amino acids include arginine, asparagine, aspartate, glutamine, glutamate, histidine, lysine, serine, and threonine. The hydrophobic amino acids include alanine, cysteine, isoleucine, leucine, methionine, phenylalanine, proline, tryptophan, tyrosine and valine. Other families of amino acids include (i) serine and threonine, which are the aliphatic-hydroxy family; (ii) asparagine and glutamine, which are the amide containing family; (iii) alanine, valine, leucine and isoleucine, which are the aliphatic family; and (iv) phenylalanine, tryptophan, and tyrosine, which are the aromatic family.

The term “agent” is used herein to denote a chemical compound, a mixture of chemical compounds, a biological macromolecule, or an extract made from biological materials.

The term patient includes human and veterinary subjects.

The disclosure also includes Fv, Fab, Fab′ and F(ab′)2 anti-CD3 antibody fragments, single chain anti-CD3 antibodies, bispecific anti-CD3 antibodies, heteroconjugate anti-CD3 antibodies, trispecific antibodies, immunoconjugates and fragments thereof.

Bispecific antibodies are antibodies that have binding specificities for at least two different antigens. In the present case, one of the binding specificities is for CD3. The second binding target is any other antigen, and advantageously is a cell-surface protein or receptor or receptor subunit.

All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same. The disclosure having now been described by way of written description, those of skill in the art will recognize that the disclosure can be practiced in a variety of embodiments and that the foregoing description and examples below are for purposes of illustration and not limitation of the claims that follow.

### EXAMPLES

### Example 1: Bioavailability Study of Foralumab Administered by Subcutaneous Delivery in a Mouse Model

The objective of this study was to compare the pharmacokinetic (PK) profiles of intravenous and subcutaneous administration of foralumab in a mouse model.

The results of this study will provide feasibility for administering foralumab via subcutaneous injection, which represents a possible human therapeutic route. The intravenous route of administration will be used as a comparator because it was validated in previous pre-clinical repeated dose toxicity studies and early clinical studies for administering foralumab.

Study Design

The mouse model used in this study was the human CD3 epsilon transgenic mouse model, which harbors a humanized CD3 epsilon chain of the CD3 co-receptor within a functional mouse immune system. This model is used to determine the in vivo efficacy of human-specific immunotherapies that target the human CD3 epsilon chain. A total of 132 mice (66 males and 66 females) were used. Groups were dosed either intravenously (IV) or by subcutaneous injection (SC). Study groups 1, 2 and 3 were composed of 18 males and 18 females, divided into groups of 3 for each time point. Intravenous (IV) group 4 had 3 males and 3 females and the subcutaneous placebo group 5 had 9 males and 9 females. For each time point, 3 males and 3 females were used as described below (Table 1.1).

TABLE 1.1
Study Groups

| Group | Control/ | Route of | | Time Points (hour) | | | | | |
|---|---|---|---|---|---|---|---|---|---|
| Number | Test Article | Injection | Gender | 0.5 | 2 | 6 | 24 | 48 | 120 |
| 1 | NI-0401 | IV | Males | 3 | 3 | 3 | 3 | 3 | 3 |
| | | | Females | 3 | 3 | 3 | 3 | 3 | 3 |
| 2 | NI-0401 | SC 1x | Males | 3 | 3 | 3 | 3 | 3 | 3 |
| | | | Females | 3 | 3 | 3 | 3 | 3 | 3 |
| 3 | NI-0401 | SC 2x | Males | 3 | 3 | 3 | 3 | 3 | 3 |
| | | | Females | 3 | 3 | 3 | 3 | 3 | 3 |
| 4 | Placebo | IV | Males | — | — | — | 3 | — | — |
| | | | Females | — | — | — | 3 | — | — |
| 5 | Placebo | SC | Males | — | — | — | 3 | 3 | 3 |
| | | | Females | — | — | — | 3 | 3 | 3 |

Dose Formulation

Foralumab (M-0401) was formulated in 25 mM sodium acetate buffer, 125 mM sodium chloride with 0.02% polysorbate 80, pH 5.5. The vehicle control (Placebo) used was 25 mM sodium acetate buffer, 125 mM sodium chloride with 0.02% polysorbate 80, pH 5.5.

Dose Level and Volume

A single dose of 0.3 mg/kg was selected which was shown previously to cause up to 70% reduction of T cells in peripheral blood and 80% modulation of human CD3 epsilon molecules at the T cell membrane in LCD3 transgenic mice. Dose volume was 2.5 mL/kg manually injected as a bolus.

Test Article Administration

A single dose of either the foralumab formulation or placebo was administered subcutaneously in the lateral side of the abdominal wall or else intravenously via the retro-orbital sinus.

Blood Sampling

Blood samples were collected from the mice by intracardiac puncture under terminal anaesthesia at the indicated time points following administration. Samples were collected in Plasma Separator Tubes (BD), and the plasma separated by centrifugation. Aliquots of 40-50 μL plasma were frozen and stored at −50° C.

Results and Conclusion

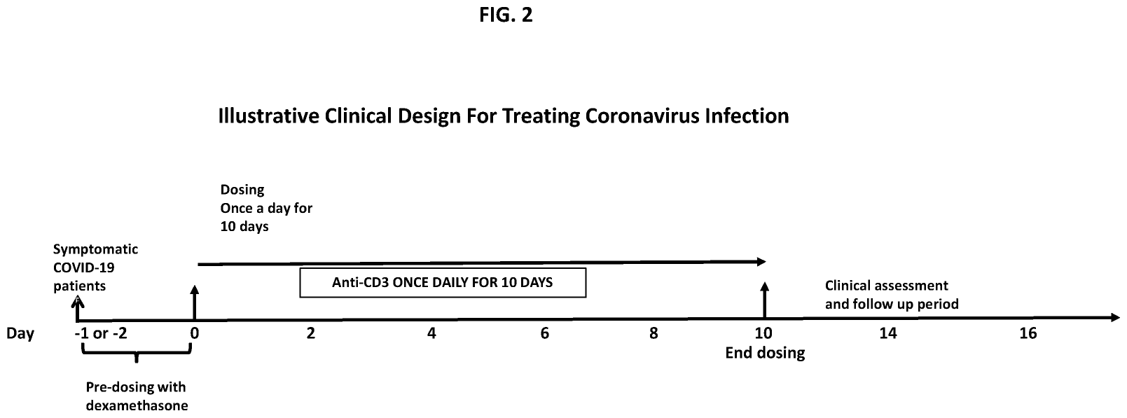

Subcutaneous administration of foralumab achieves efficient delivery into the blood and is pharmacologically active (FIG. 2, Table 1.2). Blood levels of subcutaneously delivered foralumab peaked between 6 and 24 hours following delivery (Table 1.3). Bioavailability of foralumab at 0.3 mg/kg delivered subcutaneously is approximately 55% (Table 1.2). Increasing the dose of foralumab to 0.6 mg/kg in a subcutaneous delivery increases the bioavailability to 92% (Table 1.2). Infusion related reactions are likely to be reduced because of a 50% reduction in Cmax achieved through subcutaneous compared to intravenous administration. Together these results suggest that delivery of foralumab by subcutaneous administration is a valid method for dosing subjects foralumab and related antibodies.

TABLE 1.2
Pharmacokinetic Parameters
Non Compartmental Analysis

| | | | | AUC | AUC | | Half- | | | |
|---|---|---|---|---|---|---|---|---|---|---|
| Dose | Dose | Tmax | Cmax | (0-t) | (0-inf.) | λz | life | No. | Bioavail | MRT |
| (mg/kg) | Route | (h) | (ng/mL) | (ng · h/mL) | (ng · h/mL) | (/hr) | (h) | Points | (%) | (h) |
| 0.3 | IV 1x | 0.0 | 7306 | 289439 | 476318 | 0.00749 | 93 | 3 | 100 | 127 |
| 0.3 | SC 1x | 6 | 2167 | 167001 | 261271 | 0.0085 | 81 | 4 | 55 | 118 |
| 0.6 | SC 2x | 24 | 5117 | 500505 | 873420 | 0.0072 | 96 | 3 | 92 | 142 |

TABLE 1.3
Plasma Concentrations from Different Routes of Administration

| PK Time (h) | 0.3 mg/kg IV 1x | 0.3 mg/kg SC 1x | 0.6 mg/kg SC 2X |
|---|---|---|---|
| 0 | 7306 | 0 | 0 |
| 0.5 | 7040 | 70 | 913 |
| 2 | 6283 | 950 | 3467 |
| 6 | 4660 | 2167 | 4183 |
| 24 | 3050 | 1733 | 5117 |
| 48 | 2033 | 1567 | 5000 |
| 120 | 1400 | 802 | 2683 |

### Example 2: Pharmakokinetic, Pharmacodynamics, and Safety Studies of Foralumab (NI-0401) by Intravenous Administration

The primary objective of these studies were to assess the safety and tolerability of foralumab in human subjects. The studies include an assessment of the pharmacokinetic profile, immunogenicity, and pharmacodynamic effects of foralumab delivered by intravenous administration for five days.

Test Product, Dose, and Mode of Administration

The foralumab human monoclonal antibody was supplied in 3 mL vials, each containing 2 mL of the foralumab formulation at a concentration of 2.0 mg/mL. Each vial contained 4.0 mg of foralumab. The dose of foralumab was administered by intravenous infusion over 2 hours. The dosing schedules of foralumab were provided for eight different cohorts according to Table 2.1.

TABLE 2.1
Dosing Schedule

| | Day 1 (μg/m2 | Day 2 (μg/m2 | Day 3 (μg/m2 | Day 4 (μg/m2 | Day 5 (μg/m2 |
|---|---|---|---|---|---|
| | of Body | of Body | of Body | of Body | of Body |
| Cohort | Surface Area) | Surface Area) | Surface Area) | Surface Area) | Surface Area) |
| Cohort 1 | 500 | 500 | 500 | 500 | 500 |
| Cohort 2 | 500 | 500 | 650 | 650 | 650 |
| Cohort 3 | 650 | 650 | 650 | 650 | 650 |
| Cohort 4 | 650 | 650 | 1000 | 1000 | 1000 |
| Cohort 5 | 1000 | 1000 | 1000 | 1000 | 1000 |
| Cohort 6 | 1250 | 1250 | 1250 | 1250 | 1250 |
| Cohort 7 | 1500 | 1500 | 1500 | 1500 | 1500 |
| Cohort 8 | 1750 | 1750 | 1750 | 1750 | 1750 |

Pharmacokinetic profiles were determined by measuring foralumab blood plasma levels at the indicated times following administration. Foralumab plasma levels were measured using a ligand-binding assay. A specific anti-foralumab antibody was used as capture reagent and a fluorescently labeled anti-human IgG1. The sensitivity of the assay was 20 ng/mL (lower limit of detection).

Pharmacokinetic Results

Foralumab plasma levels were measured using a ligand-binding assay. A specific anti-foralumab antibody was used as capture reagent and a fluorescently labeled anti-human IgG1. The sensitivity of the assay was 20 ng/mL (lower limit of detection).

For the study design in Table 2.1, the Cmax and AUC0-t parameters on Study Day 5 yielded mean values of 584.4 ng/mL (range 28.43 to 1860.1 ng/mL) and 28570 ng.h/mL (range 1453 to 106494 ng.h/mL), respectively across the dose range of 0.73 to 3.73 mg.

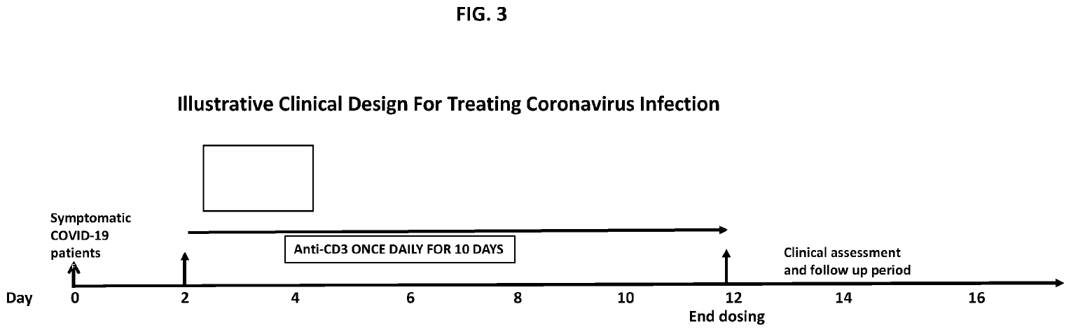

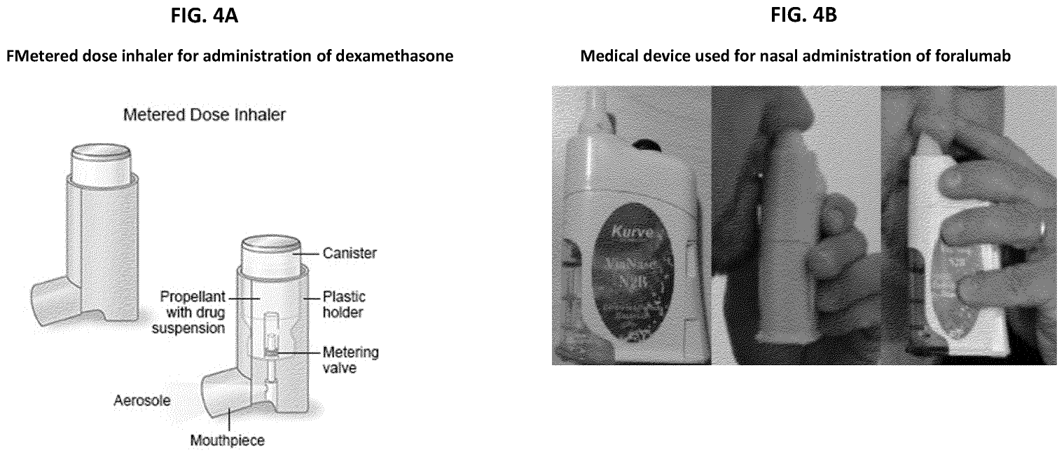

A representative sampling of pharmacokinetic profiles for three individuals from a separate study is shown in FIG. 3. These subjects were treated either with five doses of 1.0 mg (approximately +/−500 μg/m2), two doses of 2.0 mg (approximately +/−1000 μg/m2), or a single dose of 10.0 mg (approximately +/−5000 μg/m2). For these 3 subjects, the concentration profiles were adequate to estimate apparent Cmax (1 hour post infusion) and AUC0-6, both showing a clear increase with dose (FIG. 4). The apparent Cmax values for the 1.0 mg, 2.0 mg, and 10.0 mg doses were 110 ng/mL, 350 ng/mL, and 2800 ng/mL, respectively. The AUC values for the 1.0 mg, 2.0 mg, and 10.0 mg doses were 440 ng.h/mL, 1700 ng.h/mL, and 11800 ng.h/mL, respectively.

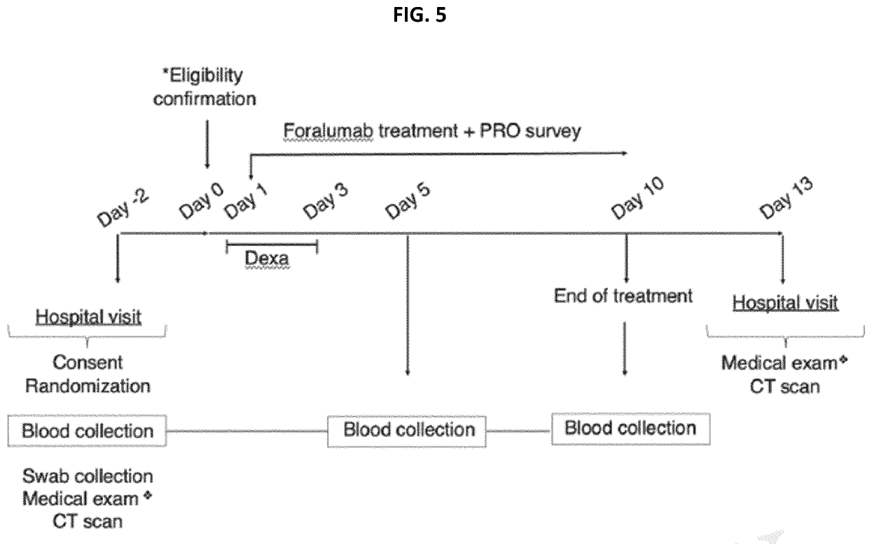

Graphs summarizing foralumab PK data obtained after a single 2 h-infusion of 10.0 mg (subject 001-0001 in FIG. 4) are presented in FIG. 5. Following intravenous infusion of 10.0 mg study drug over 2 hours, the plasma concentrations of foralumab increased rapidly during the infusion period as expected. Post infusion, the concentration values declined in an essentially mono exponential manner. The early rapid decline in plasma concentration is due to binding of the drug to its target on circulating T-cells as well as drug distribution. The estimated plasma terminal half-life after a single dose of 10.0 mg foralumab was about 13 h although with a more sensitive assay the terminal half-life may be considerably longer than this (theoretically it would take approximately 78 h to be eliminated from the systemic circulation, this being 6 times the terminal half-life). The measured half-life of foralumab is shorter than expected for an IgG1 molecule (usually approximately 3 weeks) because of the rapid uptake of the drug by the target cells.

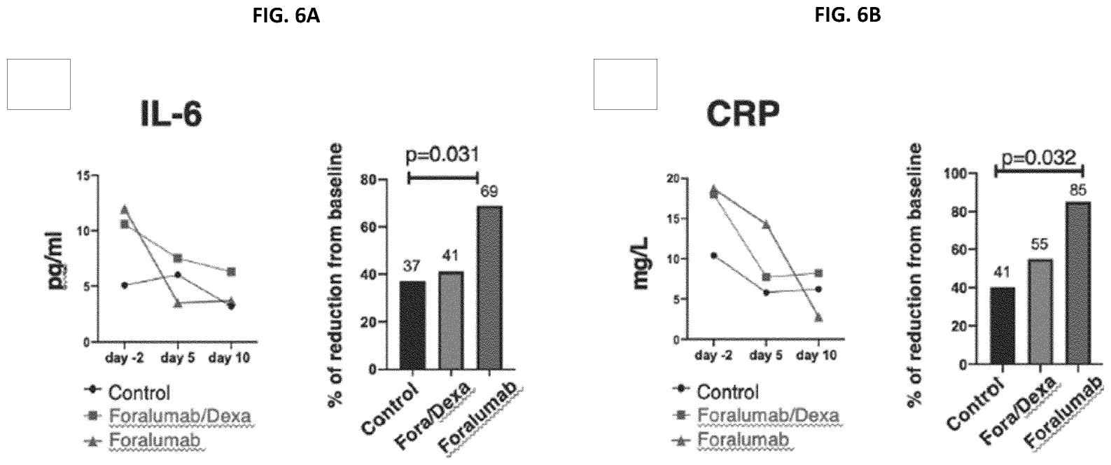

Exposure based on AUC0-t from Study Day 1 to 5 indicated there was accumulation of foralumab in the blood plasma. One subject (030-0003 in FIG. 4) who was exposed to 5 doses of 1.0 mg foralumab corresponding to approximately 21 μg/kg/dose (+/−500 μg/m2/dose) had plasma drug concentrations increasing during the treatment period (FIG. 6). When overlayed, the PK and PD profiles in this subject correlated, both showing a peak at the end of the treatment period as shown in the graph below (FIG. 6). This may be due to the frequency of dosing and/or the depletion of the target having a decreased effect on mAb disposition.

Overall these observations suggest that intravenous administration of foralumab at doses of 1.0 mg (i.e. 21 μg/kg or 500 μg/m2) and above may result in an accumulation of drug over the 5 days dosing period. However, drug is expected to be eliminated rapidly and within approximately 3-4 days of the final dose.

Pharmacodynamic Results

There was no discrimination by foralumab dose on its expected pharmacology for all pharmacodynamic analyses. All dose levels of foralumab had effects on the TCR-CD3 complex and cellular populations in the time courses observed.

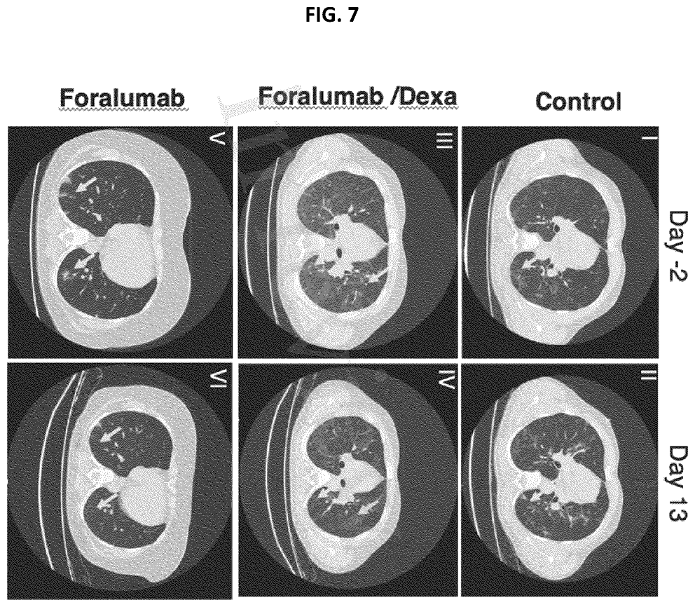

Modulation of the TCR-CD3 complex was measured in CD8+ or CD4+ T cells after the beginning of treatment at the indicated time points (FIG. 7) The maximum modulation of TCR-CD3 complex for all treatment groups was observed at the end of the treatment period on Study Day 5. The mean modulation across all treatment groups on Study Day 5 was 81.1% and the highest mean TCR-CD3 complex modulation at this point was observed in treatment cohort 8 (94%) (FIG. 8). TCR-CD3 modulation gradually decreased after the treatment period ended (FIG. 8). All patients in all treatment cohorts (for whom CD3 modulation data were available) achieved CD3 modulation above 50% (FIG. 8). CD3 modulation across all treatment groups remained above 50% for a mean duration of 8.7 days and above 30% for a mean duration of 12.9 days.

A preliminary kinetic profile of TCR-CD3 modulation per cohort (doses from 500 to 1500 μg/m2) of 3 patients is summarized in FIG. 9. The profile describes a dose dependent effect with a peak reached at day 5 followed by a gradual decline until day 21 (Week 3). The 3 highest dose regimen groups currently tested and ranging between 2 and 3 mg/day have the same profile with a mean TCR-CD3 modulation at day 10 around 50%, representing a plateau reached at those dose regimens (FIG. 9).

Circulating leucocytes and sub-population counts were generated during and after the time course of foralumab dosing. There was a transient elevation in CD45+ leucocyte count in all the foralumab treatment cohorts (overall mean 68.7% elevation) at 6 hours post-dose on Study Day 1 followed by a return to levels close to, or below baseline in most cohorts. By Week 3 the CD45+ leucocyte counts were below their baseline value in all cohorts except for 1 and 4. There was a rapid and almost complete disappearance (>90%) of CD45+ lymphocytes, CD3+ T-cells, CD3+CD4+ T-cells (helper T-cells) and CD3+CD8+ T-cells (cytotoxic T-cells) from the circulation within 24 h of first infusion in all treatment groups followed by a return close to baseline values by Week 3. These results strongly suggest foralumab can induce lymphodepletion in humans. There was no apparent dose-response in the reduction or recovery although the recovery in cohort 1 was faster than in all other cohorts, except for CD8+ cell counts. There was also a rapid decrease in CD3-CD19+(B-cell) and CD3-CD16+CD56+ cell (Natural killer cell) counts in all treatment groups 6-hours post-dose on Study Day 1, with a variable (non-dose-dependent) recovery in these cell counts at Study Day 3 and values above baseline at week 3 in the majority of cases.

Cytokine levels were assessed in subjects administered foralumab (FIG. 10). Substantial variations between subjects in their release of cytokines following treatment with foralumab were observed, which did not appear to be dose-related. In patients with notable elevations of pro-inflammatory cytokines, this was accompanied by symptoms suggestive of an infusion-related reaction (IRR), although most symptoms were mild and of short duration. Most patients had little or no evidence of pro-inflammatory cytokine release on subsequent treatment days.

Safety Results

Adverse Events (AEs) were assessed in human subjects given foralumab by intravenous administration (FIG. 11). Nineteen of the 24 patients (79%) experienced a total of 94 AEs of which 3 (3%) were serious adverse events (SAEs) SAEs. Fifty-eight AEs (62%) were of mild severity, 32 (34%) of moderate severity and 4 (4.3%) were severe. Sixty-eight out of the 91 non-serious AEs (75%) were considered by the investigators to have a reasonable possibility of being drug-related. The AEs that occurred during the 5-day treatment period were mainly defined as being infusion related reactions (IRRs) (61%) as they were reported during, or within 24 hours following, an infusion of foralumab (FIG. 12 and FIG. 13). The most common IRRs were chills (9 events in 6 patients), pyrexia (8 events in 7 patients), headache (8 events in 6 patients), hypotension (5 events in 3 patients) and elevated ALT (3 events in 3 patients).

One IRR was reported as an SAE: A patient in cohort 5 had transient elevation of ALT on Study Day 2 leading to interruption in study drug treatment. Visual inspection indicates an apparent dose-response in the reporting of treatment-related AEs, with more drug-related AEs reported by the higher dose cohorts; all the blood and lymphatic system disorders were reported by the two highest dose cohorts.

Three SAEs were reported; two were considered to be unrelated to the study drug: one patient had a relapse of Crohn's disease 8 days after completion of the 5-day treatment, resulting in prolongation of hospitalisation and a second patient had a multi-fragmental fracture of the left humeral bone with bone fragments displacement following a fall in the street, one and a half months after the end of study treatment. One SAE was considered to be related to the study drug: a transient elevation of ALT on Study Day 2 leading to interruption in study drug treatment. Few AEs (18) were reported after the 5-day treatment period during the study. There were no deaths and no AEs that led to study discontinuation.

Hematology and biochemical analysis in subjects receiving foralumab indicated that, in general, mean hemoglobin, hematocrit and red blood cell count values remained stable in all treatment cohorts over time and there was no substantial change over time in mean platelet count across the treatment cohorts. By Study Day 5 there was a reduction from baseline in mean total white blood cell count of −2.98 10E9/L across all treatment cohorts, with no evidence of a dose-response. White blood cell counts had recovered by Week 12. Mean neutrophil and monocyte counts showed the same pattern, with a mean drop on Study Day 5 and recovery by Week 12 and Week 3 respectively.

Liver function was assessed in subjects receiving foralumab and some transient and non-serious liver test abnormalities were detected (FIG. 14). Five patients (15%) had isolated and transient rise in ALP above the upper limit of normal range starting at day 5 in two cases, and at week 2, 3 and 4 in the other cases respectively. One patient had preexisting abnormal ALP levels. One patient had a transient and isolated rise in AST/ALT at 3.5 twice the upper limit of normal range at day 5 which was normalized at week 2. Six patients (18%) had liver tests abnormalities depicting a cholestatic liver injury of mild severity and transient in duration. No rise in serum bilirubin was associated except in one patient. Out of these six patients, three already had preexisting liver test abnormalities of same magnitude. The rise of hepatic enzymes occurred mainly at day 5 and returned to basal levels within one week. One patient had mild jaundice (rise in bilirubin on day 2 and resolved on day 4) and another had hepatomegaly (a second rise in liver enzymes reoccurred at week 4 in this patient who also had preexisting liver test abnormalities). No other signs of hepatic insufficiency were noted in these cases and no causal factor has been identified.

### CONCLUSIONS

From the safety data it can be concluded that the dose-limited-toxicity dose was not reached in these studies. However, the safety profile of foralumab was extended by the steroid premedication from a daily dose of 500 mg/m2 (˜1.00 mg) to 1750 mg/m2 (˜3.5 mg). This study has shown foralumab to be pharmacologically active. Foralumab had an impact on the TCR-CD3 complex and on T-cell subsets reflecting the expected pharmacology of this drug and its target. After foralumab treatment, lymphocyte counts were decreased below the normal range in all patients. There was no obvious effect of foralumab dose on the duration of lymphocyte depletion. Mean lymphocyte counts were back close to pre-treatment values by Week 4. Lymphocyte depletion is an expected effect of administration of an anti-CD3 antibody.

### Example 3: Treatment of COVID-19 with Anti-CD3 Antibody in Human Subjects

Study Design

Human subjects with flu-like symptoms consistent with COVID-19 infection were evaluated and screened for the study. Inclusion criteria comprised a positive RT-PCR COVID-19 test, 39 subjects were enrolled.

Subjects were randomized into three cohorts: no foralumab treatment (n=16), nasal foralumab and dexamethasone (n=11), and nasal foralumab alone (n=12). 50 μg of foralumab was administered by nose drop to each nostril (total 100 μg). Subjects receiving dexamethasone received 6 mg of oral dexamethasone on days 1-3. Subjects in the control group did not receive foralumab.

Laboratory Tests

Nasopharyngeal swabs were used to screen for COVID-19 by RT-PCR. Clinical laboratory tests included complete blood counts, IL-6, D-dimer, CRP, COVID-19 Serology, HIV, syphilis, pregnancy, hepatitis, and glycated hemoglobin. White blood counts were measured by flow cytometry and impedance. C-reactive protein (CRP) and glycated hemoglobin were measured by turbidimetry, D-dimer was measured by immunoturbidimetry, and IL-6 was measured by chemiluminescence.

Serum levels of IL-6, CRP and D-dimer were quantified on days −2, 5 and 10. As shown in FIG. 6A and FIG. 6B, foralumab resulted in a 69% reduction in IL-6 levels at day 10 (p=0.031) and 85% reduction in CRP at day 10 (p=0.032). As shown in Table 3.1, three group comparisons showed a difference between Control vs Foralumab at a day 5 (p=0.01) and at day 10 (p=0.031) and in CRP on day 10 (p=0.032). These results suggest treatment with Foralumab can reduce the immune hyperactivity and adverse inflammatory response associated with coronaviral infection.

TABLE 3.1
Levels of IL-6, CRP, and D-dimer

| | | | |
|---|---|---|---|
| | Day −2 | Day 5 | Day 10 |
| Treatment Group | (Mean ± SD) | (Mean ± SD) | (Mean ± SD) |

IL-6

| | | | |
|---|---|---|---|
| Control | 5.1 (±5.4) | 6 (±7.5) | 3.2 (±1.9) |
| Foralumab/Dexa | 10.6 (±9.2) | 7.5 (±9.6) | 6.3 (±9.2) |
| Foralumab | 12 (±11.5) | 3.5 (±3) | 3.7 (±2.4) |

IL-6 Statistical Analysis

| | | | |
|---|---|---|---|
| Three group comparison (p-value) | 0.09 | 0.41 | 0.29 |
| †Control - Foralumab/Dexa | — | 0.20 | 0.40 |
| †Control - Foralumab | — | 0.01 | 0.031 |
| †Foralumab - Foralumab/Dexa | — | 0.42 | 0.032 |

| | | |
|---|---|---|
| (Mean ± SD) | (Mean ± SD) | (Mean ± SD) |

CRP

| | | | |
|---|---|---|---|
| Control | 10.4 (±19.6) | 5.8 (±12.8) | 6.2 (±12.2) |
| Foralumab/Dexa | 18 (±25.7) | 7.7 (±22.1) | 8.2 (±15.3) |
| Foralumab | 18.7 (±24.5) | 14.3 (±18.7) | 2.8 (±3.6) |

CRP Statistical Analysis

| | | | |
|---|---|---|---|
| Three group comparison (p-value) | 0.56 | 0.44 | 0.52 |
| †Control - Foralumab/Dexa | — | 0.27 | 0.57 |
| †Control - Foralumab | — | 0.87 | 0.032 |
| †Foralumab - Foralumab/Dexa | — | 0.23 | 0.26 |

| | | |
|---|---|---|
| (Mean ± SD) | (Mean ± SD) | (Mean ± SD) |

D-dimer

| | | | |
|---|---|---|---|
| Control | 368.8 (±156.7) | 888.6 (±1635.6) | 573.5 (±436.8) |
| Foralumab/Dexa | 434.6 (±216.8) | 555.5 (±376.6) | 417.1 (±210.4) |
| Foralumab | 499.5 (±317.3) | 757.7 (±872.5) | 551.8 (±356.5) |

D-dimer Statistical Analysis

| | | | |
|---|---|---|---|
| Three group comparison (p-value) | 0.34 | 0.77 | 0.52 |
| †Control - Foralumab/Dexa | — | 0.34 | 0.35 |
| †Control - Foralumab | — | 0.28 | 0.64 |
| †Foralumab - Foralumab/Dexa | — | 0.56 | 0.92 |

Lung and CT analysis

Lung CT scan was performed using a 16 channel (Toshiba-Alexion) CT scanner. Contrast was not used. Scan coverage was from apex of the lung to the level of bilateral adrenals. Tube voltage was between 100-120Kv. Parenchyma slide thickness was 1 mm.

Lung injury consisted of patchy shadowing and ground glass appearance which was graded on a scale of 0 to 4 as follows: 0=no detectable abnormalities or lung involvement <5%; Stage 1=mild lung involvement involving approximately 10% of lung area, Stage 2=moderate lung involvement with patchy shadowing and ground glass lesions involving approximately 25% of lung area, Stage 3=severe confluent ground glass lesions and consolidation involving 25% to 50% of lung area; Stage 4 very severe ground glass lesions and consolidation involving more than half of the lung area.

Foralumab treatment was given for 10 consecutive days. Computerized tomography (CT) of the lung was obtained prior to treatment at day −2 and at study completion on day 13 and analyzed. Baseline lung CT scans were compared to scans obtained on day 13. Subjects were classified as worsened if they increased by one or more stage, improved if they decreased by one stage and as having marked improvement if they decreased by 2 or more stages. Subjects were stable if they did not change stages. Lung CT analysis was performed by three radiologists in a blinded fashion.

Results from the Lung CT scan are shown in FIG. 7. Axial images in a control patient shows widespread ground glass opacity (anterior and posterior segments of bilateral upper and right middle lobes) two days prior to treatment (I) showing significant progression at 13 days follow up (II). Axial images in a patient treated with foralumab and dexamethasone (Foralumab/Dexa) showing both widespread ground glass opacity in the anterior and posterior segments and consolidation in both lower lobes (III) demonstrating partial resolution on the 13 follow up day scan (IV). V-VI: Axial images in a patient who received foralumab showing ground glass opacity of posterior segments of lungs (V) demonstrating interval resolution on 13 follow up day scan (VI).

Each patient was classified as worse, stable, improved or markedly improved. As shown in Table 3.2, 1/10 of the foralumab+dexamethasone treated subjects worsened. 10/14 control subjects, 2/10 foralumab+dexamethasone subjects and 2/12 foralumab subjects remained stable. Regarding improvement, because 6 patients in the control group and 2 in the foralumab+dexamethasone group had no lung involvement on day −2, they were not able to improve. Improvement occurred in 3/8 control, 1/8 foralumab+dexamethasone, and 5/12 in the foralumab group. Marked improvement was observed in 1/8 control, 6/8 in the foralumab+dexamethasone and 5/12 in the foralumab group. Thus, marked improvement was predominantly observed in subjects receiving foralumab+dexamethasone or foralumab alone. Control vs. foralumab+dexamethasone, p=0.01 and control vs. forlumab/dexamethasone+foralumab, p=0.04 (chi-square analysis).

TABLE 3.2
Lung Injury Assessment

| | | Foralumab + | |
|---|---|---|---|
| | Control | Dexamathasone | Foralumab |
| Worsened | 0/14 | 1/10 | 0/12 |
| Stable | 10/14 | 2/10 | 2/12 |
| Improved | 3/8 | 1/8 | 5/12 |
| Marked Improvement | 1/8 | 6/8 | 5/12 |

Patient Reported Outcomes (PRO) and Medical Report Outcome

PRO consisted of 15 questions with the following response system: (1) Anosmia (loss of smell): 0=normal, 3=reduced, 5=completely lost). (2) Dysgeusia (loss of taste): 0=normal, 3=reduced, 5=completely lost). (3) Cough: 0=not present, 3=present some time, 5=present more than half the day. (4) Headache: 0=not present, 3=present some time, 5=present more than half the day); (5) Throat ache: 0=not present, 3=moderate, hurts when swallowing, 5=strong, intense pain when swallowing. (6) Dyspnea: 0=not present, 3=moderate, some lack of air, 5=strong, difficult breathing. (7) Nausea/Vomiting: 0=not present, 3=nausea without vomiting, 5=vomiting. 8) 02 Saturation: 0=>95, 3=94-95%, 5=91-93%. (9) Diarrhea was evaluated according to the Bristol Scale (ref) 0=type 0-4, 3=type 5 or 6, 5=type 7. (10) Rhinorrhea: 0=not present, 3=nose with mucus, 5=runny nose (liquid)); (11) Abdominal pain: 0=not present, 3=moderate, 5=intense). (12) Myalgia: 0=not present, 3=moderate, 5=intense (full body). (13) Fever: 0=not present, 3=37-38C, 5=>38.0C. (14) Conjunctivitis: 0=absent, 5=present. (15) Appetite: 0=normal, 3=reduced, 5=completely lost. General well-being (how you are feeling today) was assessed using the Baker Wong scale for pain assessment (0-10). The maximum possible score was 85.

COVID-19 symptoms reported at day −2 were compared to symptomatology at day 13. Subject reported symptoms were stratified according to Domains as follows: Domain 1 (weakness, fatigue, inappetence, body ache, backpain); Domain 2 (fever, chills, sweating); Domain 3 (nausea, diarrhea, epigastric pain); Domain 4 (ageusia); Domain 5 (anosmia); Domain 6 (runny nose, odynophagia, sneezing); Domain 7 (headache, anxiety, eye pain, dizziness); Domain 8 (cough, dyspnea, chest pain). Each subject was scored for one symptom in each domain.

At day −2, subjects had been experiencing symptoms for an average of 6 days in an average of 5 Domains. Most subjects improved during the course of the study with no major differences between the treatment groups. At the end of the study 23 of 39 subjects (58.9%) were asymptomatic; 8 of 16 (50%) in the control group, 6 of 11 (54.5%) in the foralumab+dexamethasone group and 9 of 12 (75%) in the foralumab group. Among the 16 subjects that remained symptomatic at the end of the study, anosmia (Domain 5) and cough (Domain 8) were the most common symptoms. There were anecdotal reports of rapid recovery from anosmia and ageusia in both foralumab treated groups. These results suggest intranasal administration of foralumab can improve COVID-19 symptoms.

### OTHER EMBODIMENTS

While the disclosure has been described in conjunction with the detailed description thereof, the foregoing description is intended to illustrate and not limit the scope of the disclosure, which is defined by the scope of the appended claims. Other aspects, advantages, and modifications are within the scope of the following claims.

We claim:

1. A method of treating, preventing or alleviating a symptom of a coronavirus infection in a subject in need thereof comprising administering to the subject a composition comprising an anti-CD3 antibody.

2. The method of claim 1, where the antibody is a monoclonal antibody.

3. The method of claim 1 or 2, wherein the antibody is fully human or humanized.

4. The method of any one of claims 1 to 3, wherein the anti-CD3 antibody comprises a heavy chain complementarity determining region 1 (CDRH1) comprising the amino acid sequence GYGMH (SEQ ID NO: 42), a heavy chain complementarity determining region 2 (CDRH2) comprising the amino acid sequence VIWYDGSKKYYVDSVKG (SEQ ID NO: 43), a heavy chain complementarity determining region 3 (CDRH3) comprising the amino acid sequence QMGYWHFDL (SEQ ID NO: 44), a light chain complementarity determining region 1 (CDRL1) comprising the amino acid sequence RASQSVSSYLA (SEQ ID NO: 45), a light chain complementarity determining region 2 (CDRL2) comprising the amino acid sequence DASNRAT (SEQ ID NO: 46), and a light chain complementarity determining region 3 (CDRL3) comprising the amino acid sequence QQRSNWPPLT (SEQ ID NO: 47).

5. The method of any one of claims 1 to 4, wherein the anti-CD3 antibody comprises a variable heavy chain amino acid sequence comprising the amino acid sequence of SEQ ID NO: 48 and a variable light chain amino acid sequence comprising the amino acid sequence of SEQ ID NO: 49.

6. The method of any one of claims 1 to 5, wherein the anti-CD3 antibody comprises a heavy chain amino acid sequence comprising the amino acid sequence of SEQ ID NO: 50 and a light chain amino acid sequence comprising the amino acid sequence of SEQ ID NO: 51.

7. The method of any one of claims 1 to 6, wherein the coronavirus is SARS-CoV, SARS-CoV-2, MERS-CoV, or a mutant or a variant thereof.

8. The method of any one of claims 1 to 7, wherein the symptom of a coronavirus infection is one or more of hyperactive immune response, fever, gastrointestinal symptoms, respiratory symptoms, anosmia (loss of smell), dysgeusia (loss of taste), cough, headache, throat ache, pain when swallowing, dyspnea, difficult breathing, shortness of breath, nausea, vomiting, reduced O2 saturation, diarrhea, rhinorrhea, abdominal pain, myalgia, fever, conjunctivitis, and loss of appetite.

9. The method of claim 8, wherein the hyperactive immune response comprises increased levels of at least one of interleukin 6 (IL-6), C-reactive protein (CRP), and D-dimer.

10. The method of claim 9, wherein the levels of at least one of IL-6, CRP, and D-dimer are reduced.

11. The method of any one of claims 1 to 10, wherein the subject has or is suspected of having a coronavirus infection.

12. The method of any one of claims 1 to 11, wherein the subject has been or thought to have been exposed to a coronavirus and has not yet developed symptoms of a coronavirus infection.

13. The method of any one claims 1 to 12, further comprising administering to the subject a composition comprising dexamethasone.

14. The method of any one of claims 1 to 13, wherein the composition is administered orally, mucosally, by inhalation, nasally, intravenously or any combination thereof.

15. The method of claim 14, wherein the inhalation administration is by an inhaler or a nebulizer.

16. The method of any one of claims 1 to 15 further comprising administering an anti-TNFα antibody, an anti-CD20 antibody an anti-IFNγ antibody, an anti-Granulocyte-Macrophage Colony-Stimulating Factor antibody or an anti-IL-6R antibody.

17. The method of any one claims 1 to 16, further comprising administering to the subject an antiviral drug, an immune booster drug, vitamin C, Vitamin D, Vitamin E or any combination thereof.

18. The method of claim 17, wherein the antiviral drug is Azidothymidine, Remdesivir or Actinomycin D.

19. The method of any one claims 1 to 18, wherein the anti-CD3 is administered nasally at a daily dose of 50 μg to 100 μg.

20. The method of claim 19 wherein the daily dose is administered once daily.

21. The method of claim 19 or 20 wherein the daily dose is administered for at least 10 consecutive days.

22. The method of any one of claims 1 to 18, wherein the anti-CD3 is administered orally at a daily dose of 1.0 to 2.5 mg.

23. The method of any one of claims 18 to 22 wherein the subject is further administered dexamethasone.

24. The method of claim 23 wherein the dexamethasone is administered by inhalation.

25. The method of claim 24, wherein the administration by inhalation by a metered inhaler.

26. A nasal or inhalation formulation comprising an anti-CD3 antibody and dexamethasone.

27. A method of treating, preventing, or alleviating a symptom of a disease or disorder in a subject in need thereof comprising administering to the subject a composition comprising an anti-CD3 antibody.

28. The method of claim 27, wherein the subject has an inflammatory disease or disorder.

29. The method of claim 28, wherein the inflammatory disease or disorder is autoimmune encephalomyelitis, lupus, or arthritis.

30. The method of claim 27, wherein the subject has a pulmonary disease or disorder.

31. The method of claim 30, wherein the subject the pulmonary disease or disorder is acute respiratory distress syndrome (ARDS).

32. The method of claim 27, wherein the subject has a neurodegenerative disease.

33. The method of claim 31, wherein the subject has a neurodegenerative disease or disorder.

34. The method of claim 32, wherein the neurodegenerative disease or disorder is multiple sclerosis.

35. The method of claim 33, wherein the multiple sclerosis is secondary progressive multiple sclerosis.

36. A method of treating, preventing or alleviating a symptom of a disease or disorder in a subject in need thereof comprising:
a. Collecting a sample from the subject;
b. Measuring a marker for hyperactive immune response;
c. Administering to the subject a composition comprising an anti-CD3 antibody based on a level of the marker.

37. The method of claim 36, wherein the disease or disorder is a coronavirus infection, an inflammatory disease or disorder, a pulmonary disease or disorder, or a neurodegenerative disease or disorder.

38. The method of claim 36 or 37, wherein the marker for hyperactive immune response is at least one of interleukin 6 (IL-6), C-reactive protein (CRP), D-dimer, interferon (IFN), interferon alpha (IFN-α), interferon gamma (IFN-γ), interleukin 1 beta (IL-1β), and/or CXCL10.

39. The method of any one of claims 36 to 38, wherein the level of the marker is elevated or high compared to a healthy subject.

40. The method of any one of claims 36 to 39, wherein the anti-CD3 antibody is foralumab.
